(19) United States
(12) Patent Application Publication
(10) Pub. No.: US 2023/0351111 A1
(43) Pub. Date: Nov. 2, 2023

(54) SVO ENTITY INFORMATION RETRIEVAL SYSTEM

(71) Applicant: BenevolentAI Technology Limited, London (GB)

(72) Inventor: Julien FAUQUEUR, London (GB)

(73) Assignee: BenevolentAI Technology Limited, London (GB)

(21) Appl. No.: 17/786,922

(22) PCT Filed: Dec. 9, 2020
(86) PCT No.: PCT/GB2020/053156

§ 371 Date: Jun. 17, 2022

(30) Foreign Application Priority Data
Dec. 20, 2019 (GB) 1919111.3

(51) Int. Cl. G06F 40/295; G06F 40/242; G06F 16/31; G16B 40/00; G16B 50/10

(52) U.S. Cl. CPC G06F 40/295; G06F 40/242; G06F 16/316; G16B 40/00; G16B 50/10

(57) ABSTRACT

Methods, apparatus, system and computer-implemented method are provided for a computer-implemented method of automatically extracting entities associated with one or more domain(s) of interest from a corpus of text. A plurality of portions of text are received from the corpus of text, each portion of text comprising data representative of at least two entities and/or relationships thereto. For each received portion of text, identifying one or more subject-verb-object (SVO) entity data item(s) comprising data representative of at least two entities, a relationship associated with the at least two entities, a subject entity corresponding to an entity of said at least two entities, an object entity corresponding to an entity of the at least two entities, a verb portion associated with the relationship, and a direction of the relationship associated with the at least two entities. A graph structure based on the set of identified SVO entity data items is output, the graph structure comprising a graph of entity nodes and relationship edges linking the entity nodes with each relationship edge including an indication of directionality of said relationship.

The present application relates to a system and method for retrieving Subject-Verb-Object entity information via one or more Subject-Verb-Object entity data items.

### BACKGROUND

In drug discovery, pertinent biomedical or biological relationships between entities or entities of interest such as, by way of example only but not limited to, a drug and a disease are important clues for identifying a potential blockbuster drug. Therefore, methods to extract and verify these relationships through analysing the text of documents are extremely valuable. Statistics-based methods such as co-occurrence counts have traditionally been used for this purpose. However, these methods have a high chance of missing much of the contextual information that relates various entities and relationships thereto such as contextual information related to biological entities of interest including, without limitation, for example drug to the disease, disease to target, disease to protein/gene, disease to mechanism/process, and protein/gene to protein/gene, target to target and other entities of interest. More information regarding the entities of interest is typically required when researching the pertinent biological or biomedical relationships between entities.

Natural language processing (NLP) is a technique applicable to analyse data stored in a corpus of text that includes, by way of example only but not limited to, text, documents, patents, research papers, and/or other literature within one or more domains of interest. NLP can provide automated processing of the corpus of text and extraction of any relevant information thereof. More specifically, NLP can extract semantic information pertaining to the entities of interest by analysing the text in a high-throughput manner. Indeed, this avoids significant reliance on experts to review the contents of the corpus of text. However, textual information extracted via NLP tends not to be further characterised to yield the pertinent or relevant information of relationships between entities associated with one or more domains of interest. For example, entity relationships or paths are not further categorised via many present day NLP approaches.

Thus, it has been found that when using automated methods of, for example, drug discovery, methods used for extracting relationships are a key tool for identifying entities that are candidates for new biomedical relationships, or verifying existing relationships via additional relationships, classifying document contents or indeed any other method that uses the related entities detected in the documents. However, simple methods such as co-occurrence counts or any other statistics-based metric has a high chance of missing much of the information contained in the relationship statements that relate the entities such that more information about how they are related may be extracted.

There is a desire for a mechanism or apparatus capable of automatically retrieving the pertinent and/or relevant information of entity relationships between entities from portions of text from a corpus of text and efficiently and concisely outputting a data structure of this information for use by researchers and/or other system(s) in a workflow associated with, for example, drug discovery and the like.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter; variants and alternative features which facilitate the working of the invention and/or serve to achieve a substantially similar technical effect should be considered as falling into the scope of the invention disclosed herein.

The present disclosure provides method(s), system(s) and apparatus for, in response to a search query associated with entities or a domain of interest, automatically processing text portions of a corpus of text associated with a domain of interest to generate search results. The text portions are processed by identifying and extracting entities and relationships thereto associated with the search query, analysing the entities and relationships thereto, and identifying subject entity, object entity and verb portion of the relationship and extracting contextual information such as, without limitation, for example direction of the relationship, entity sign of the relationship and other meta-data. This search result information is output as a graph structure with a plurality of entity nodes and relationship edges therebetween, with the subject and object entity data, verb portion and contextual information embedded within the graph structure forming an enhanced set of search results that include the most pertinent and relevant information associated with the entities and relationships thereto.

In a first aspect, the present disclosure provides a computer-implemented method of automatically extracting entities associated with one or more domain(s) of interest from a corpus of text, the method comprising: receiving a plurality of portions of text from the corpus of text, each portion of text including data representative of at least two entities and/or relationships thereto; identifying, for each received portion of text, one or more subject-verb-object (SVO) entity data item(s) including data representative of at least two entities, a relationship associated with the at least two entities, a subject entity corresponding to an entity of said at least two entities, an object entity corresponding to an entity of the at least two entities, a verb portion associated with the relationship, and a direction of the relationship associated with the at least two entities; outputting a graph structure based on the set of identified SVO entity data items, the graph structure comprising a graph of entity nodes and relationship edges linking the entity nodes with each relationship edge including an indication of directionality of said relationship.

As an option, the computer-implemented method further including identifying meta-data from each of the received text portions for inclusion to each SVO entity data item, the meta-data comprising data representative of one or more from the group of: directionality associated with each relationship; biological sign or entity sign, where applicable, associated with each relationship; affirmation or negation information associated with each relationship; context information associated with each relationship; any other contextual data associated with said each relationship; and any other contextual data associated with the directionality and/or biological sign associated with each relationship; and outputting the graph structure based on the set of identified SVO data items, wherein the relationship edges linking the entity nodes include indications of the one or more identified meta-data from the corresponding SVO entity data item(s) associated with the entity nodes.

As an option, each of the at least two entities comprise data representative of a noun or a noun phrase associated with the one or more domains of interest. As an option, the subject entity corresponds to a first noun or a first noun phrase and the object entity corresponds to a second noun or a second noun phrase. Optionally, each entity of the at least two entities is a named entity from an entity dictionary associated with at least one of the domain(s) of interest.

As an option, identifying one or more SVO entity data items comprises identifying the first and second entities as named entities from the portion of text based on one or more entity dictionaries associated with said one or more domains of interest.

As an option, identifying the first and second entities further comprises performing an entity search of the received portions of text based on the one or more entity dictionaries associated with the one or more domain(s) of interest for identifying data representative of at least two entities associated with the one or more domains of interest and an entity dependency relationship therebetween.

As an option, the computer-implemented method further comprising building a graph search index comprising the output graph structure.

As an option, identifying an SVO entity data item for each received portion of text further comprising performing relationship extraction on said each received text portions to identify at least two entities and an entity dependency relationship therebetween.

As an option, receiving the plurality of portions of text from the corpus of text, further comprising performing relationship extraction on the received portions of text for at least predicting or identifying at least two entities and an entity dependency relationship thereto.

As an option, receiving the plurality of portions of text from the corpus of text, further comprising: receiving a plurality of portions of text from the corpus of text; and detecting, from the received plurality of portions of text, one or more portions of text likely to include at least one entity for use in identifying SVO entity data.

As an option, identifying an SVO entity data item for each of the received portions of text further comprising performing SVO identification on said each received text portions based on identifying: a subject entity corresponding to an entity of the at least two identified entities; an object entity corresponding to an entity of the at least two identified entities; and a verb portion associated with the identified relationship.

As an option, performing SVO identification further comprising: detecting linguistic features of the from each of the received portions of text that connect the at least two identified entities; extracting data representative of the subject entity, object entity, verb portions, and direction based on the at least two identified entities; and adding the extracted direction indication to the relationship associated with the at least two entities.

As an option, identifying SVO entity data item(s) further comprising performing meta-data identification on each of the received text portions based on determining data representative of one or more from the group of: an indication of the direction of the identified relationship between said at least two entities based on identified subject and object entities; biological sign/entity sign, if any, of the identified relationship between said at least two entities based on identified subject and object entities; affirmation or negation information associated with the identified relationship corresponding to said at least two entities based on identified subject and object entities; context information associated with the identified relationship between the at least two identified entities based on identified subject and object entities; and any other contextual data associated with the relationship between one or more of the at least two identified entities, identified subject entity, identified object entity, verb portion and/or direction; and wherein the SVO entity data item further comprises data representative of the identified meta-data.

As an option, performing SVO identification for each received portion of text further comprising: detecting linguistic features from one or more segments of text of the received portion of text that connect the at least two identified entities; and extracting data representative of the subject entity, object entity, verb portions, and direction based on the detected linguistic features from said segments and the at least two identified entities.

As an option, identifying SVO data items(s) further comprising: performing SVO entity identification on each of the received text portions based on identifying a subject entity, an object entity, and a verb entity associated with a relationship between the identified subject entity and the identified object entity; performing relationship extraction on each of the received text portions to identify at least two entities and an entity dependency relationship therebetween; and associating the subject entity with one of the at least two identified entities, the object entity with one of the at least two identified entities, and the verb entity identifying an entity of the at least two identified entities to the subject-entity.

As an option, identifying, from each of the received portions of text, SVO entity data representative of at least two entities and a relationship associated with the at least two entities further comprising: inputting each received portion of text into a relationship extraction model configured for predicting or identifying at least two entities and a relationship therebetween for said each received portion of text.

As an option, identifying, from each of the received portions of text, SVO entity data representative of a subject entity corresponding to an entity of the at least two entities, an object entity corresponding to an entity of the at least two entities, a verb portion associated with the relationship, further comprising: inputting at least two entities and a relationship therebetween in relation to each received portion of text into a SVO extraction model configured for predicting or identifying a subject entity corresponding to an entity of the at least two entities, an object entity corresponding to an entity of the at least two entities, a verb portion associated with the relationship therebetween for said each received portion of text.

As an option, identifying, from each of the received portions of text, SVO entity data item(s) further comprising: inputting each received portion of text into a SVO identification model configured for predicting or identifying a subject entity corresponding to an entity of the at least two entities, an object entity corresponding to an entity of the at least two entities, a verb portion associated with the relationship therebetween for said each received portion of text.

As an option, for each SVO entity data item, identifying the subject entity and object entity as an entity pair. As an option, for each SVO entity data item, identifying the at least two identified entities as an entity pair.

As an option, the method further comprising: determining whether any duplicate SVO entities exist within the set of SVO entities; and removing any duplicate SVO entities from the set of SVO entities. As an option, the domain of interest includes biological and/or chemical domains of interest and the entities have entity types in the domain of biological and/or chemical domains. As an option, method further comprising receiving a selection of one or more domain(s) of interest.

As an option, identifying, for each of the received portions of text, an SVO entity data item further comprising: identifying one or more SVO triples based on the at least two entities and an entity dependency relationship therebetween, wherein the subject of one of the SVO triples is associated with a first entity of the at least two entities, the object of said one of the SVO triples is associated with a second entity of the at least two entities, and the verb of said one of the SVO triples is associated with the entity dependency relationship between the first and second entities; and determining, for each identified SVO triple, meta-data representative of at least the direction of the entity dependency relationship between the first and second entities corresponding to said each SVO triple; and outputting an SVO entity data item comprising data representative of the identified SVO triple and at least the direction of the entity dependency relationship between the first and second entities of said identified SVO triple.

As an option, identifying an SVO entity data item for each of the received portions of text further comprising: inputting said each received portion of text into an entity extraction engine configured for detecting and extracting a portion of text including at least two entities corresponding to the one or more domain(s) of interest and an entity dependency relationship therebetween; and outputting entity extraction search results comprising data representative of the extracted portion of text comprising at least two identified entities and the relationship therebetween.

As an option, the entity extraction engine or process is configured to perform the steps of: identifying, from the corpus of text, candidate portions of text including one or more entities of interest corresponding to the domain(s) of interest; detecting the most likely candidate portions of text containing at least two entities and an entity relationship therebetween; extracting data representative of the detected entities and relationships therebetween from the detected candidate portions of text; and outputting data representative of entity search results based on the extracted data representative of entities and relationships therebetween.

As an option, detecting the most likely candidate portions of text further comprises parsing each identified candidate portion of text to determine whether an entity relationship exists in relation to the one or more entities.

As an option, the entity extraction engine or process comprises an entity extraction machine learning model configured to identify, predict, detect and/or extract portions of text comprising at least two entities associated with the one or more domains of interest and a relationship therebetween from a corpus of text or documents.

As an option, inputting portions of text from the corpus of text associated with the one or more domain(s) of interest to one or more machine learning, ML, extraction model(s) configured for identifying and/or predicting whether the portions of text include at least two entities in one or more domain(s) of interest and an entity dependency relationship therebetween.

As an option, inputting portions of text determined to include one or more entity(ies) associated with one or more domain(s) of interest to one or more machine learning, ML, extraction model(s) configured for identifying and predicting whether a portion of text with one or more entity(ies) of interest forms at least two entities and an entity dependency relationship therebetween.

As an option, the entity extraction engine or process further comprises a rule-based engine or process configured to: identify, from the received portions of text of the corpus of text, text portions including one or more entity(ies) associated with the one or more domains of interest based an entity search of the received portions of text using on one or more entity dictionaries associated with the one or more domains of interest; and extract, from each identified text portion, data representative of at least two entities associated with the one or more domains of interest and an entity relationship therebetween.

Optionally, the step of identifying, for each of the received portions of text, one or more SVO entity data item(s) further comprising: parsing said each received portion of text for detecting linguistic features associated with the at least two entities associated with the domain(s) of interest and corresponding entity dependency relationship therebetween; identifying, from said each received portion of text, a first entity of the at least two entities associated with the subject of the received portion of text, a second entity of the at least two entities associated with the object of the received portion of text, and a verb segment of the entity dependency relationship associated with the verb of the identified relationship in the received portion of text; and outputting a set of SVO entity data items representative of an subject-verb-object triple based on data representative of the first entity, segment of the entity relationship, and the second entity.

As an option, parsing said each received portion of text for detecting linguistic features further comprising a linguistic detection engine coupled to an entity repository and an entity relationship repository, wherein the linguistic detection engine is configured to use one or more entity repositories in the domain(s) of interest and entity relationship repositories to process said each received portion of text by: detecting linguistic features in said each received portion of text associated with a first entity and a second entity of at least two entities and the entity dependency relationship therebetween; and identify the first entity as the subject, the second entity as the object and a segment of the entity dependency relationship as the verb of said each received portion of text.

As an option, determining, for each SVO entity data, at least the biological sign and direction of the entity dependency relationship based on a domain mapping engine coupled to an ontological dictionary of relational terms associated with entities and entity relationships, the domain mapping engine configured for: determining a segment of the entity relationship representing a biological/entity sign of the entity dependency relationship for the at least two entities of said each SVO entity data item; determining a direction indication of the entity dependency relationship representing the direction of the entity dependency relationship between the first and second entities of the at least two entities of said each SVO entity data item; and updating said each SVO entity data item with data representative of the segment representing the biological/entity sign of the entity dependency relationship and data representative of the direction indication of the entity dependency relationship.

As an option, determining one or more further contextual elements of the entity relationship representing the context of the entity relationship between the first and second entities of the at least two entities of said each SVO entity data item; and updating said each SVO entity data item representative of the contextual segments.

As an option, determining, for each identified SVO entity data item, at least the biological sign, and direction of the entity relationship based on: inputting data representative of a received portion of text associated with the SVO entity data item, the corresponding at least two entities, and/or the corresponding entity relationship, to a domain mapping machine learning model configured to identify or predict a biological sign of the entity dependency relationship for the at least two entities, and to identify or predict a direction indication of the entity relationship representing the direction of the entity relationship between the first and second entities of the at least two entities; and updating said each SVO entity data item with data representative of the predicted biological sign and direction of the entity relationship.

As an option, storing data representative of each of the output identified SVO entity data item(s) and corresponding biological sign and direction of the entity relationship based on: performing validation, conflict resolution and/or aggregation of the plurality of identified SVO entity data item(s) for input to an SVO search index data structure based on one or more from the group of: new SVO entity data items; any contradicting SVO entity data items; multiple identical SVO entity data items that are the same; multiple SVO data items with identical first and second entities with different relationships; and storing the validated SVO entity data items in the SVO search index data structure for use in outputting SVO search results based on received SVO search queries querying the SVO search index data structure, wherein the SVO search queries comprise data representative of one or more entities, process(es) and/or relationships thereto in the domain(s) of interest.

As an option, aggregating two or more of the identified SVO entity data items(s) with the same entity pair and similar entity relationship by: aggregating the biological sign indications associated with the two or more identified SVO entity data item(s) to determine an overall biological sign; aggregating the direction indications associated with the two or more identified SVO entity data item(s) to determine an overall direction indication; generating an aggregated SVO entity data item comprising data representative of the entity pair, the entity dependency relationship, and the overall biological sign and overall direction indication; and storing data representative of the aggregated SVO data item in the SVO search index data structure.

As an option, the SVO search index data structure comprises a graph structure based on the output and/or stored set of SVO entity data item(s).

As an option, the set of SVO entity data items comprise a plurality of SVO entity data items, each SVO entity data item associated with an indication of the biological sign and direction of the entity relationship between at least two entities, and the set of SVO entity data items are stored in a graph structure comprising a plurality of nodes linked together by edges, wherein each node of the graph structure represents an entity, and an edge linking a pair of nodes represents a relationship between a pair of entities represented by the pair of nodes, the edge further comprising data representative of an indication of the direction associated with the relationship between the pair of entities.

As an option, receiving a search query comprising data representative of one or more entities, process(es), and/or relationships thereto associated with one or more domain(s) of interest; querying the graph structure for finding a relevant set of nodes and/or edges associated with the search query, and outputting a sub-graph of the graph structure based on the relevant set of nodes and/or edges associated with the search query.

As an option, querying the graph structure for determining whether SVO data items exist in the graph structure associated with the search query; in response to determining SVO entity data items exist, generating a knowledge sub-graph associated with the plurality of entities based on one or more of: SVO entity data items output from the graph structure in relation to the search query; filtering the SVO knowledge graph based on the search query; in response to determining SVO entity data items in relation to the search query are non-existent or are out-of-date, performing the steps of receiving portions of text from the corpus of text, identifying SVO entity data items, and outputting/storing data representative of the sets of SVO entity data items for updating the graph structure.

As an option, a search query comprises a request for a labelled training dataset associated with entity pairs and relationships thereto associated with domain(s) of interest, wherein the method further comprising: processing the SVO entity data items output from the SVO search index data structure in relation to the search query into a labelled training dataset, wherein the labelled training dataset is for use as an input labelled training dataset for training one or more ML model(s) associated with predicting or classifying objective problems and/or processes in the field of: biology, biochemistry, chemistry, medicine, chem(o)informatics, bioinformatics, pharmacology, and any other field relevant to diagnostic, treatment, and/or drug discovery and the like; and sending the processed SVO entity data items as a labelled training dataset in response to the request. As an option, the labelled training dataset comprises a labelled graph structure.

As an option, a biological and/or chemical entity comprises entity data associated with an entity type from at least the group of: gene; disease; compound/drug; protein; cell type; tissue; chemical; organ; biological parts; mechanisms or systems; or any other entity type associated with bioinformatics, chem(o)informatics, biology, biochemistry, chemistry, medicine, pharmacology, and/or any other field relevant to diagnostic, treatment, and/or drug discovery and the like.

In a second aspect, the present disclosure provides a computer-readable medium comprising code or computer instructions stored thereon, which when executed by a processor unit, causes the processor unit to perform the computer-implemented method according to any one of the features, steps, process(es) of the first aspect, combinations thereof, modifications thereto, and/or as herein described.

In a third aspect, the present disclosure provides an apparatus comprising a processor unit, a memory unit and a communication interface, the processor unit connected to the memory unit and communication interface, wherein the apparatus is adapted to implement the computer-implemented method according to any one of the features, steps, process(es) of the first aspect, combinations thereof, modifications thereto, and/or as herein described.

In a fourth aspect, the present disclosure provides an SVO apparatus of automatically extracting entities associated with one or more domain(s) of interest from a corpus of text, the system comprising:an input module configured to receive a plurality of portions of text from the corpus of text, each portion of text comprising data representative of at least two entities and/or relationships thereto; an SVO engine configured to identify, for each received portion of text, one or more subject-verb-object “SVO” entity data items comprising data representative of at least two entities, a relationship associated with the at least two entities, a subject entity corresponding to an entity of the at least two entities, an object entity corresponding to an entity of the at least two entities, a verb portion associated with the relationship, and a direction of the relationship associated with the at least two entities; and an output module configured to output a set of identified SVO entity data items.

As an option, the output module is further configured to outputting a graph structure based on the set of identified SVO entity data items, the graph structure comprising a graph of entity nodes and relationship edges linking the entity nodes with each relationship edge including an indication of directionality of said relationship.

As an option, the output module further configured to build a graph search index based on the graph structure, the graph search index comprising a graph of entity nodes with relationship edges between each entity and an indication of the verb portion and directionality associated with each relationship between entities.

As an option, the SVO apparatus is adapted to implement the computer-implemented method according to any one of the features, steps, process(es) of the first aspect, combinations thereof, modifications thereto, and/or as herein described.

In a fifth aspect, the present disclosure provides a search system, the system comprising: a search query module configured for receiving a search query comprising data representative of one or more entities and/or relationships associated with one or more domains of interest; an SVO search module configured for processing the search query based on an SVO search index data structure; and an SVO apparatus configured or adapted according to any of the features, steps, process(es) of the first, second, third or fifth aspects, the SVO apparatus configured for building or updating the SVO search index data structure based on an output set of SVO entity data items.

Optionally, the first, second, third, fourth, and/or fifth aspects, where the corpus of text comprises a large scale document repository including a plurality of documents associated with a plurality of domain(s) of interest, biological entity and/or chemical entity concepts and the like.

Optionally, the first, second, third, fourth, and/or fifth aspects, where the corpus of text comprises data representative of one or more from the group of: unstructured text, semi-structured text, documents, sections of documents, sentences and/or paragraphs of documents, tables, and/or any portions of text and/or data representative of one or more entities and/or relationships thereto capable of being detected and/or identified using relationship extraction techniques and the like.

Optionally, the first, second, third, fourth, and/or fifth aspects, where an entity comprises entity data associated with an entity type in relation to a domain of interest from at least the group of: bioinformatics; chem(o)informatics; data informatics; social media; entertainment; geographical; any other entity type in which a portion of text comprises data representative of a relationship for one or more entity(ies).

Optionally, the first, second, third, fourth, and/or fifth aspects, where the domain of interest comprises one or more domains or fields associated with an entity type from at least the group of: genes; diseases, disease process(es) or pathway(s); biological part(s), biological process(es) or pathway(s); compound/drug; protein(s); cell-line(s); chemical; tissue;

organ; or any other domain of interest or entity type associated with bioinformatics, pharmacology and/or chem(o)informatics and the like.

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

The features of each of the above aspects and/or embodiments may be combined as appropriate, as would be apparent to a skilled person, and may be combined with any of the aspects of the invention. Indeed, the order of the embodiments and the ordering and location of the preferable features is indicative only and has no bearing on the features themselves. It is intended for each of the preferable and/or optional features to be interchangeable and/or combinable with not only all of the aspect and embodiments, but also each of preferable features.

### BRIEF DESCRIPTION OF THE DRAWINGS

Embodiments of the invention will be described, by way of example, with reference to the following drawings, in which:

FIG. 1a is a flow diagram illustrating an example subject-verb-object (SVO) identification process according to the invention;

FIG. 1b is a flow diagram illustrating another example SVO identification process according to the invention;

FIG. 1c is a flow diagram illustrating a further example SVO identification process according to the invention;

FIG. 1d is a flow diagram illustrating another example SVO identification process according to the invention;

FIG. 1e is a flow diagram illustrating yet another example SVO identification process according to the invention;

FIG. 2a is a flow diagram illustrating an example process for extracting entities and/or associated relationships from a corpus of text thereto use by the SVO identification processes of FIGS. 1a to 1e according to the invention;

FIG. 2b is a schematic diagram illustrating an entity extraction system based on the process of FIG. 2a using machine learning techniques according to the invention;

FIG. 2c is a schematic diagram illustrating an entity extraction system based on the process of FIG. 2a using rule-based and entity dictionary techniques according to the invention;

FIG. 3a is a flow diagram illustrating an example process for extracting entity relationships from text portions including entity pairs for use by the SVO identification process(es) of FIGS. 1a to 1e according to the invention;

FIG. 3b is a schematic diagram illustrating an entity relationship extraction system based on the process of FIG. 3a using machine learning techniques according to the invention;

FIG. 3c is a schematic diagram illustrating an entity relationship extraction system based on the process of FIG. 3a using rule-based and relationship dictionary techniques according to the invention;

FIG. 4a is a flow diagram illustrating an example SVO process for generating subject-verb-object triples from entity pairs and associated entity dependency relationships according to the invention;

FIG. 4b is a schematic diagram illustrating an example SVO labelling for an text portion of an entity pair of interest based on the SVO process of FIG. 4a according to the invention;

FIG. 4c is a schematic diagram illustrating an SVO search engine based on the process of FIG. 4a using machine learning technique(s) according to the invention;

FIG. 4d is a schematic diagram illustrating an SVO search engine based on the process of FIG. 4a using rule-based and/or relationship, sign and/or direction dictionary techniques according to the invention;

FIG. 4e is a schematic diagram illustrating an example SVO entity data item and knowledge graph representation for input to and/or output from an SVO database or repository according to the invention;

FIG. 5a is a flow diagram illustrating an SVO search process in relation to system of FIG. 5ba according to the invention;

FIG. 5b is a system diagram illustrating an example search system based on FIG. 5a according to the invention;

FIG. 6 is a schematic diagram illustrating an example SVO knowledge graph for biological and/or chemical entities of interest retrieved from the system of FIG. 1a according to the invention;

FIG. 7a is a schematic diagram illustrating a computing system and device according to the invention; and

FIG. 7b is a schematic diagram illustrating a system according to the invention.

Common reference numerals are used throughout the figures to indicate similar features.

### DETAILED DESCRIPTION

Embodiments of the present invention are described below by way of example only. These examples represent the best mode of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples. For the avoidance of any doubt, the features described in any embodiment are combinable with the features of any other embodiment and/or any embodiment is combinable with any other embodiment unless express statement to the contrary is provided herein. Simply put, the features described herein are not intended to be distinct or exclusive but rather complementary and/or interchangeable.

The present invention is related to an end-to-end process and system for identifying and extracting entities associated with one or more domain(s) of interest from a corpus of text automatically using a SVO workflow (e.g. SVO process, engine or apparatus). In particular, the SVO workflow receives a plurality of portions of text, e.g. a sentence or paragraph, from the corpus of text associated with one or more domains of interest. Each portion of text may include data representative of at least two entities and/or relationships thereto that may be identified and/or extracted. These entities and/or relationships are analysed to determine subject, verb and/or object data associated with the entities and/or relationships for establishing further information contained in the relationship statements that relate the entities such that more information about how they are related may be extracted. This information is identified and extracted from text portions using the SVO workflow for outputting data representative of an identified set of SVO entity data items based on the received text portions from the corpus of text. Each SVO entity data item of the identified set of SVO entity data items may include data representative of at least two entities, a relationship associated with the at least two entities, a subject entity corresponding to a first entity of the at least two entities, an object entity corresponding to a second entity of the at least two entities, and enhanced relationship information including, without limitation, for example a verb portion associated with and/or concisely describing the relationship, an indication of the sign/direction of the relationship and/or any other meta-data of contextual data associated with the at least two entities. The identified set of SVO entity data items be output in the form of, without limitation, for example a graph structure. For example, a graph structure including a of entity nodes (e.g. each entity from the set of SVO entity data items) and relationship edges linking the entity nodes with each relationship edge including enhanced relationship information including, without limitation, for example an indication of directionality of said relationship and/or biological sign of said relationship and the like.

Thus, by detecting linguistic features in the portions of text (e.g. sentences, phrases, paragraphs, text segments and the like) enables meta-data on the portions of text to be determined for representing dependency paths, directionality of the relationship to be determined (e.g. whether the relationship is positive or negative with regards to how the two entities are related), biological signs/entity signs and/or information to be determined, affirmation or negation information to be determined, and/or any other meta-data and/or context information between subject entities, object entities, relationships thereto, such that more detailed relationship identification, extraction and representation can take place. Representations of the enhanced or more detailed relationship information associated with at least two entities can then be used for, without limitation, for example drug discovery and/or optimisation workflows and the like.

Each of the SVO entity data items includes data representative of the enhanced or more detailed relationship information for at least two entities and relationship thereto for each portion of text of a plurality of portions of text from the corpus of text. Thus the set of SVO data items may be used and/or efficient represented, without limitation, for example in graph structures with the enhanced relationship information represented as labelled edges connecting entity nodes, which represent the entities identified from the portions of text; used to build search index graphs and/or knowledge graphs with said relationship data/information used in edges connecting the entity nodes of a search index graph/knowledge graph and the like. These efficient representations of the set of SVO entity data items are beneficial to, without limitation, for example processes and/or workflows in drug discovery, drug optimisation, and/or used to generate drug hypotheses from identified entity pairs and/or relationships thereto that are thought to be related based on the connections of the graph structures representing the set of SVO entity data items.

Identifying one or more SVO data items may further include identifying meta-data from each of the received text portions for inclusion as, without limitation, for example enhanced relationship information into each SVO entity data item. The identified meta-data may include data representative of one or more from the group of, without limitation, for example: directionality associated with each entity relationship of the SVO entity data item; biological/entity sign, where applicable, associated with each entity relationship of the SVO entity data item; affirmation and/or negation information associated with each entity relationship of the SVO entity data item; context information associated with each entity relationship of the SVO entity data item; any other contextual data associated with said each entity relationship of the SVO entity data item; and any other contextual data associated with the directionality and/or biological sign associated with each entity relationship of the SVO entity data item. This enhanced relationship information may be efficiently represented in relationship edges connecting entity nodes of a graph structure based on said set of SVO entity data items. Alternatively or additionally, affirmation or negation information associated with each entity relationship of the SVO entity data item may be associated with the entity itself (entity-level negation) or in the case when identifying, for each received portion of text, one or more SVO entity data item(s) comprising data representative of at least two entities (relationship-level affirmation or relationship-level negation). In the former case, when no genes could be found to interact with gene A, entity-level negation is exhibited. In the latter case, gene A does not interact with gene B shows a relationship-level negation. Moreover, the biological or entity sign may be a label suggesting a positive or negative relationship between said two entities based on identified subject and object entities. Although biological sign is used and described herein, this is for simplicity and by way of example only and the invention is not so limited, it is to be appreciated by the skilled person that the term biological sign may be applicable to any type of entity and so may be defined or used as an “entity” sign comprising or representing data representative of a label suggesting a positive or negative relationship between said two entities based on identified subject and object entities and the like. The concept of biological sign used herein may be generalised to an entity sign or specificised to an <entity-type> sign based on the entity types (or even domains) of the subject and object entities and the positive/negative relationship thereto.

Positive or negative relationships and/or associations with entities may be determined using affirmation or negation information by analysing the relationship between entities and/or terms phrases leading an entity and the like. Affirmation or negation information (or linguistic affirmation information or linguistic negation information) may comprise or represent the ways that grammar encodes negative and positive polarity in, without limitation, words, phrases, concepts, sentences, verbs, verb phrases, clauses, or other text segments and the like. For example, negation information may include, by way of example only but is not limited to, direct linguistic negation, which may include simple terms encapsulating negative polarity such as, without limitation, for example, “no”, “not”, “is not”, “cannot”, “does not”, “will not” and/or any other negative term or word, phrase and the like; or indirect linguistic negation, which may include phrases or concepts that encapsulate negative polarity and/or may have a specific negative meaning within a domain of interest such as, by way of example only but is not limited to, concepts that have a domain specific negative meanings in a domain of interest. For example, in the biological domain concepts such as “knock down”, or “silencing” or “suppression” for genes means expression of genes is reduced, “knock out” or “missing” for genes means genes are removed or not there. For example, the phrases “Missing [gene] results in [disease]” versus “Knock down of [gene] results in [disease]”, where “missing” and “knock down of are indirect linguistic negations describing a specific negative concepts with different meanings in the biological domain associated with an entity or entities. For example, affirmation information may include, by way of example only but is not limited to, direct linguistic affirmation, which may include simple terms encapsulating positive polarity such as, without limitation, for example, “yes”, “is”, “does”, “has”, “having”, “it is”, “can” and/or any other positive term or word, phrase and the like; or indirect linguistic affirmation, which may include phrases or concepts that encapsulate positive polarity and/or may have a specific positive meaning within a domain of interest such as, by way of example only but is not limited to, concepts that have a domain specific positive meanings in a domain of interest. For example, in the biological domain concepts such as “upregulating”, or “silencing” or “suppression” for genes means expression of genes is increased, “knock in” for genes means genes are replaced rather than deleted or removed. For example, the phrases “knock-in of [gene] results in [disease]” versus “upregulating of [gene] results in [disease]”, where “knock-in of” and “upregulating of” are indirect linguistic affirmations describing a specific positive concepts with different meanings in the biological domain associated with an entity or entities.

Direct and indirect linguistic affirmation or negation information in relation to an entity may lead (occur prior to the entity in a text portion) or follow (occur after the entity in a text portion). Thus, affirmation, negation and/or biological mapping in general do not always lead or follow with direct descriptive common terms, singulars, adverbs, verbs and the like, but also by indirect descriptions of other terms or phrases and concepts that may have specific meanings in domains of interest and the like, such as, without limitation, for example with ‘missing’ or “knock out” in the biological domain. Although negation information is used in examples of the invention for use in SVO data items, storing and/or using negation information associated with entities and relationships thereto, in relation to negative entity relationships, and/or relationship-level negation, and/or entity level negation (or sign) and the like, this is by way of example only and the invention is not so limited, it is to be appreciated by the skilled person that, where applicable, affirmation information such as, without limitation, for example affirmation information associated with entities and/or relationships thereto, affirmation information in relation to positive entity relationships, relationship-level affirmation, entity-level affirmation, and the like may be similarly used in SVO data items, stored and/or used as the application demands.

An advantage of the present invention pertains to the configurations of the end-to-end SVO process and system(s) described herein for outputting a graph structure based on the set of SVO entity data items with enhanced relationship information, which can be efficiently used for recognising patterns in the resulting dependency graph. The end-to-end process and system achieve this by using a separate mapping to extract direction, affirmation, negation, sign and context information in the form of SVO data entity items. In effect, the end-to-end process and system provide systematic extraction of the complete SVO information or information associated with the entities of interest separately mapped to the SVO data entity items.

Some of these approaches in the biomedical domain may include, for example, the extraction of SVO patterns and creating multi-relational ontologies and/or graph structures. The use of sign of a biological relationship may have been suggested for disparate text retrieval purposes. However, there has yet been an application or system to retrieve SVO entity information that may be used to deduce entity dependency paths associated with the SVO entity data items.

For example, the graph structure may be based on a graph structure that includes a graph of nodes linked by edges, where each node represents an entity and each edge between nodes represents a relationship between the entities represented by the nodes. Each relationship includes data indicative of the sign/direction of the relationship between the entities represented by the nodes. That is the graph structure includes entity nodes with relationship edges between each entity node, where each relationship edge includes an indication of the verb portion, sign/directionality, and/or any other meta-data associated with each relationship between entities. The graph structure may be used to update and/or build a graph search index, which may be used to output graph based search results based on search queries associated with entities, entity concepts and the like within one or more domains of interest.

A domain or domain may comprise or represent a field, subject-matter area or expert area or topic that is of interest to a user. For example, a domain or domain of interest used in examples of the present invention may include one or more domains, fields or subject-matter areas from the group of, by way of example only but is not limited to, bioinformatics; medicine; pharmacology and/or chem(o)informatics and/or any other domain, field or subject-matter area associated with drug discovery and the like. Other domains of interest may be applicable such as, by way of example only but not limited to, data informatics, social media; entertainment; financial news, financial reports; geographical data fields and the like.

An entity type may comprise or represent a label or name given to a set of entities associated with a domain that may be grouped together and share one or more characteristics, rules and/or properties and/or are considered to be listed under the same entity type. For example, in domains such as, by way of example only but not limited to, the bioinformatics and/or chem(o)informatics fields entity types may include at least one entity type from the group of, by way of example only but is not limited to, gene, genomics, gene expression and the like; anatomical region or entity; biological pathway, biological process, disease, human disease and the like; antibiotic resistance; compound/drug; protein; tissue;

cell; cell-line, or cell type; chemical; organ; food; biological; biomedical; or any other biological or biomedical entity type and the like; or any other entity type of interest associated with the bioinformatics or chem(o)informatics domains and the like. In the data informatics domains or fields and the like, an entity type may include, by way of example but not limited to, at least one entity type from the group of: news, entertainment, sports, games, family members, social networks and/or groups, emails, transport networks, the Internet, Wikipedia pages, documents in a library, published patents, databases of facts and/or information, and/or any other information or portions of information or facts that may be related to other information or portions of information or facts and the like.

An entity or entity of interest may comprise or represent an object, item, word or phrase, piece of text, or any portion of information or a fact from a portion of text and the like that may be associated with a particular entity type and be associated with a relationship. An entity or entity of interest may be, by way of example only but is not limited to, any portion of information or a fact that has a relationship, or a fact that has a relationship with another entity or entity of interest, by way of example only but is not limited to, one or more portions of information or another one or more facts and the like. An entity of interest may also comprise or represent any entity that is of interest to a user and the like. For example, in the biological, chem(o)informatics or bioinformatics domain(s) an entity of interest may comprise or represent an entity based on an entity type such as, by way of example only but is not limited to, a disease, gene, protein, compound, chemical, drug, biological pathway, biological process, anatomical region or entity, tissue, cell-line, or cell type, mechanism, disease mechanism, disease-specific mechanism, biological process, or disease process, target, or any other biological or biomedical entity and the like. In the biological domain and the like, a mechanism is a method, process or way that causes events or makes things happen within the context of biology. Mechanisms in a biological domain may include, without limitation, for example, biological processes, disease mechanisms, disease-specific processes, processes affecting biological parts, systems, tissues, and/or any other one or more process(es) within the context of the biology or biological domain and the like. For example, a biological entity of the biological entity type may be represented by data representative of an object, word or phrase from a portion of text that describes or is descriptive of that biological entity type based on the context of the text portion or text in which that entity resides. A biological entity may include entity data corresponding to a biological entity type associated with the biological domain based on, by way of example only but not limited to, one or more entity types from the group of: gene; disease; compound/drug; protein; cell type; tissue; chemical; organ; biological parts; target; disease process(es); mechanisms or systems; or any other entity type associated with bioinformatics, chem(o)informatics, biology, biochemistry, chemistry, medicine, pharmacology, and/or any other field relevant to diagnostic, treatment, and/or drug discovery and the like. An example of biological parts may be a sequence of DNA encoding a biological function from sources such as http://parts.igem.org/Help:Parts.

For example, entities of interest may be stored using, by way of example only but not limited to, graph structures, knowledge graphs and the like. As an example, entities of interest associated with a disease or gene entity type(s) may be represented using, by way of example only but not limited to, graph structures, knowledge graphs and the like, which may be based on a disease or gene ontology. Each node at a certain level in the disease or gene ontology graph describes an entity of interest at a certain level of genericity or specificity, where each parent node (or one or more ancestor node(s)) describes the entity of interest more generically, and each child node (or one or more descendant node(s)) describes the entity of interest more specifically. Example ontologies for specific biological entities may include, by way of example only but are not limited to, one or more gene ontologies for entity(ies) of the gene entity type such as, by way of example only but are not limited to, Gene Ontology (GO) from the Gene Ontology Consortium, GENIA ontology (e.g. xGENIA)—GENIA ontology may further include relationships between genes, and the like; one or more disease ontologies for entity(ies) of the disease entity type such as, by way of example only but are not limited to, “The Disease Ontology” (DO) from Northwestern University, Center for Genetic Medicine and the University of Maryland School of Medicine, Institute for Genome Sciences; one or more biological/biomedical entity ontologies or any other entity ontology based on, by way of example only but not limited to, the ontologies from the Open Biological and Biomedical Ontology (OBO) Foundry, which includes ontologies such as, by way of example only but not limited to, the Protein Ontology (https://www.ncbi.nlm.nih.gov/pmc/articles/PMC3013777/), or any type of ontology based on those from the Ontology Lookup Service (OLS) from European Molecular Biology Laboratory-European Bioinformatics Institute (EMBL-EBI), which includes ontologies associated with biological/biomedical entity types including, by way of example only but not limited to, gene, genomics, gene expression and the like; anatomical entities; disease, human disease and the like; antibiotic resistance; compound/drug; protein; tissue; cell; chemical; organ; food; biological; biomedical; or any other entity type associated with bioinformatics or chem(o)informatics and the like.

A large scale dataset, corpus of data or text associated with one or more domains of interest may comprise or represent any information, text or data from one or more data source(s), content source(s), content provider(s) and the like in relation to said one or more domains of interest. The large-scale data set or corpus of data/text, herein referred to as a corpus of text, may include, by way of example only but is not limited to, unstructured data/text, one or more unstructured text, semi-structured text, documents, sections of documents, sentences and/or paragraphs of documents, tables, structured data/text, a body of text, articles, patents and/or patent applications, publications, journals, internet (web) pages, literature, text, email, images and/or videos, or any other information or data that may contain a wealth of information corresponding to one or more domain(s) of interest and the like. This data may be generated by and/or stored with or by one or more sources, content sources/providers, or a plurality of sources (e.g. PubMed, MEDLINE, Wikipedia, US Patent Office databases, European Patent Office databases and/or any other patent data bases) and which may be used to form the corpus of text from which entities, entity types and entity relationships may be identified and/or extracted and the like. For example portions of text from the corpus of text (e.g. sentences, paragraphs, sections or segments of data from the corpus of text) may be retrieved and processed for identifying, detecting and/or extracting one or more entities and/or relationships thereto. A portion of text may describe an entity relationship associated with one or more entity(ies) and/or entity(ies) of interest associated with a domain of interest. The portion of text may be processed to identify, detect and/or extract, by way of example only but not limited to, a) one or more entity(ies) of interest associated with a domain of interest, each of which may be separable entities of interest; and b) one or more relationship entity(ies) that form and/or define the relationship associated with the one or more entity(ies) of interest, which may be separable.

Such large scale datasets or corpus of data/text may include data or information from one or more data sources, where each data source may provide data representative of a plurality of unstructured and/or structured text/documents, documents, articles or literature and the like. Although most documents, articles or literature from publishers, content providers/sources have a particular document format/structure, for example, PubMed documents are stored as XML with information about authors, journal, publication date and the sections and paragraphs in the document, such documents are considered to be part of the corpus of data/text. For simplicity, the large scale dataset or corpus of data/text is described herein, by way of example only but is not limited to, as a corpus of text.

Machine learning (ML) technique(s) may be used, by the SVO process and/or engine, to generate ML one or more model(s) for processing one or more portions of text retrieved from a corpus of text associated with one or more domains of interest and to identify SVO entity data items output from the SVO engine, and/or output as the set of SVO entity data items in the form of a graph structure or search index data structure and the like. ML techniques may use labelled training dataset(s) for use in training one or more ML model(s) (associated with predicting or classifying objective problems and/or processes in the field of: biology, biochemistry, chemistry, medicine, chem(o)informatics, bioinformatics, pharmacology, and any other field relevant to diagnostic, treatment, and/or drug discovery and the like). For example, the one or more ML model(s) may be configured for identifying and predicting whether the portions of text include at least two entities of interest associated with one or more domain(s) of interest and an entity relationship therebetween and the extraction thereof. Further, one or more ML model(s) may be configured for identifying (predicting) and/or extracting SVO data items from the portions of text of from corpus of text corresponding to one or more domain(s) of interest. Each SVO data item includes a subject entity, verb entity(ies) of the entity relationship, and object entity, and any contextual data associated with the relationship therebetween such as, affirmation and/or negation, directionality, biological sign and/or other meta-data and the like may be identified and/or extracted. For example, the sign and direction may be predicted independently by the ML model(s) or jointly with another process, i.e. ruled-based. Thus, one or more ML model(s) may be configured (e.g. in an SVO workflow) to process text portions from a corpus of text associated with one or more domains of interest and output data representative of a set of SVO data items. The identified set of SVO data items may be used for generating or updating graph structures such as graphs, knowledge graphs, building graph search index for entity search queries and the like.

ML technique(s) may further comprise or represent one or more or a combination of computational methods that can be used to generate analytical models, classifiers and/or algorithms that lend themselves to solving complex problems such as, by way of example only but is not limited to, generating embeddings, prediction and analysis of complex processes and/or compounds; classification of input data in relation to one or more relationship pertaining one or more domain(s) of interest. The one or more domain(s) of interest may comprise at least one genes; diseases, disease process(es) or pathway(s); biological part(s), biological process(es) or pathway(s); compound/drug; protein(s); cell-line(s); chemical; organ; or any other entity type associated with bioinformatics, pharmacology and/or chem(o)informatics and the like.

Examples of ML techniques that may be used by the invention as described herein may include or be based on, by way of example only but is not limited to, any ML technique or algorithm/method that can be trained on a labelled and/or unlabelled datasets to generate an embedding model, ML model or classifier associated with the labelled and/or unlabelled dataset, one or more supervised ML techniques, semi-supervised ML techniques, unsupervised ML techniques, linear and/or non-linear ML techniques, ML techniques associated with classification, ML techniques associated with regression and the like and/or combinations thereof. Some examples of ML techniques may include or be based on, by way of example only but is not limited to, one or more of active learning, multitask learning, transfer learning, neural message parsing, one-shot learning, dimensionality reduction, decision tree learning, association rule learning, similarity learning, data mining algorithms/methods, artificial neural networks (NNs), deep NNs, deep learning, deep learning ANNs, inductive logic programming, support vector machines (SVMs), sparse dictionary learning, clustering, Bayesian networks, reinforcement learning, representation learning, similarity and metric learning, sparse dictionary learning, genetic algorithms, rule-based machine learning, learning classifier systems, and/or one or more combinations thereof and the like.

Some examples of supervised ML techniques may include or be based on, by way of example only but is not limited to, ANNs, DNNs, association rule learning algorithms, a priori algorithm, Éclat algorithm, case-based reasoning, Gaussian process regression, gene expression programming, group method of data handling (GMDH), inductive logic programming, instance-based learning, lazy learning, learning automata, learning vector quantization, logistic model tree, minimum message length (decision trees, decision graphs, etc.), nearest neighbour algorithm, analogical modelling, probably approximately correct learning (PAC) learning, ripple down rules, a knowledge acquisition methodology, symbolic machine learning algorithms, support vector machines, random forests, ensembles of classifiers, bootstrap aggregating (BAGGING), boosting (meta-algorithm), ordinal classification, information fuzzy networks (IFN), conditional random field, anova, quadratic classifiers, k-nearest neighbour, boosting, sprint, Bayesian networks, Naïve Bayes, hidden Markov models (HMMs), hierarchical hidden Markov model (HHMM), and any other ML technique or ML task capable of inferring a function or generating a model from labelled training data and the like.

Some examples of unsupervised ML techniques may include or be based on, by way of example only but is not limited to, expectation-maximization (EM) algorithm, vector quantization, generative topographic map, information bottleneck (IB) method and any other ML technique or ML task capable of inferring a function to describe hidden structure and/or generate a model from unlabelled data and/or by ignoring labels in labelled training datasets and the like. Some examples of semi-supervised ML techniques may include or be based on, by way of example only but is not limited to, one or more of active learning, generative models, low-density separation, graph-based methods, co-training, transduction or any other an ML technique, task, or class of supervised ML technique capable of making use of unlabelled datasets and labelled datasets for training (e.g. typically the training dataset may include a small amount of labelled training data combined with a large amount of unlabelled data and the like.

Some examples of artificial NN (ANN) ML techniques may include or be based on, by way of example only but is not limited to, one or more of artificial NNs, feedforward NNs, recursive NNs (RNNs), Convolutional NNs (CNNs), autoencoder NNs, extreme learning machines, logic learning machines, self-organizing maps, and other ANN ML technique or connectionist system/computing systems inspired by the biological neural networks that constitute animal brains and capable of learning or generating a model based on labelled and/or unlabelled datasets. Some examples of deep learning ML technique may include or be based on, by way of example only but is not limited to, one or more of deep belief networks, deep Boltzmann machines, DNNs, deep CNNs, deep RNNs, hierarchical temporal memory, deep Boltzmann machine (DBM), stacked Auto-Encoders, and/or any other ML technique capable of learning or generating a model based on learning data representations from labelled and/or unlabelled datasets. Other examples of deep learning ML may include the use of one or more types of transformers. The transformers may be associated with the processing of natural languages, such as the Bidirectional Encoder Representations Transformers (BERT).

It is to be appreciated by the skilled person that one or more ML technique(s) may be used to generate one or more ML models, in which the one or more ML model(s) may be used in an SVO workflow to identify from a plurality of portions of text from a corpus of text, one or more SVO data item(s) as described herein and output an graph structure based on the set of SVO data items. The graph structure may be used to, by way of example only but not limited to, update a search index data structure for use in fulfilling search queries from users and the like, provided as results to a user during the drug discovery and/or optimisation process, and/or used by a drug discovery and/or optimisation workflow and the like. It will be appreciated and understood by the skilled person that the ML techniques that generate one or more ML model(s) as described and/or used herein may be applicable to operating on any corpus of text or literature, any type or entity type of one or more entity(ies) of interest, relationships and/or subject-matter thereto, and/or so as the application demands.

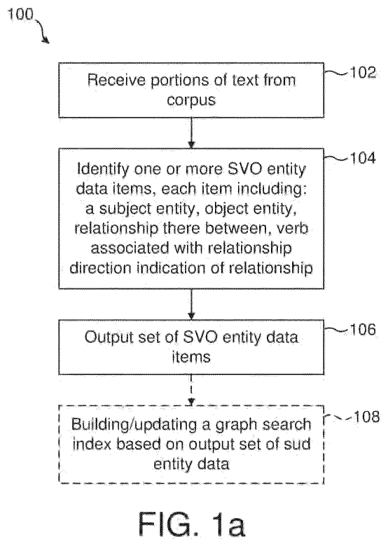

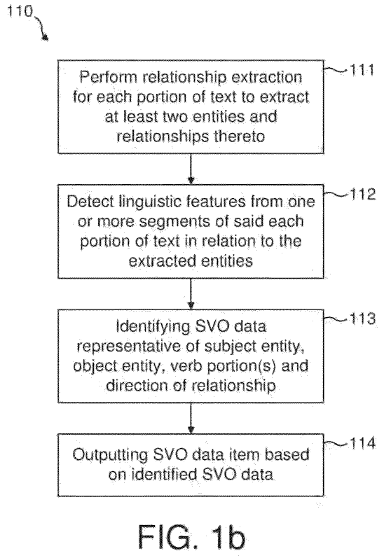

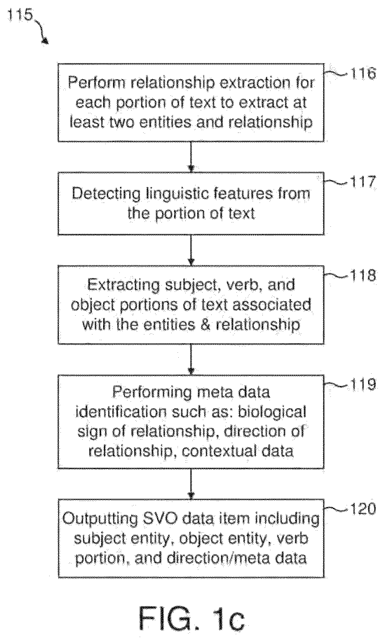

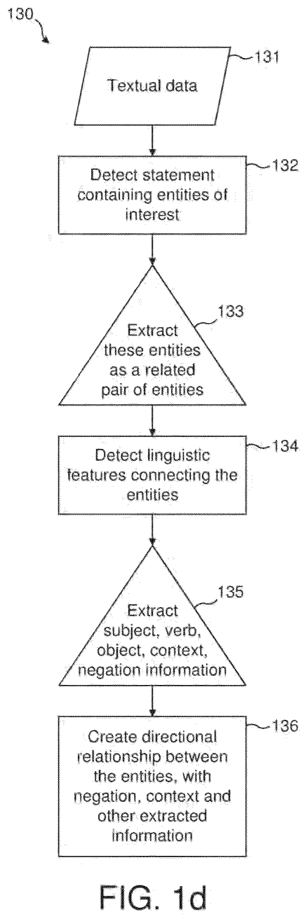

FIG. 1a is a flow diagram illustrating an exemplary subject-verb-object (SVO) process 100 for automatically identifying and/or extracting entities associated with one or more domain(s) of interest from a corpus of text according to the invention. The SVO process 100 includes the following one or more steps of: In step 102, receiving a plurality of portions of text (e.g. sentences, paragraphs, sections of text, etc.) from the corpus of text, where each portion of text includes data representative of at least two entities and/or relationship(s) thereto. In step 104, identifying, for each received portion of text, one or more SVO entity data items, each item including: a subject entity, object entity, and entity relationship therebetween, one or more verb entity(ies) associated with the entity relationship, and an indication of the direction of the entity relationship associated with the at least two entities. Each SVO entity data item(s) that is identified may include data representative of at least two entities, an entity relationship associated with the at least two entities, a subject entity corresponding to a first entity of the at least two entities, an object entity corresponding to a second entity of the at least two entities, a verb portion or entity(s) associated with the entity relationship, and/or a direction indication of the entity relationship associated with the at least two entities. In step 106, outputting data representative of a set of identified SVO entity data item(s). For example, in step 106, outputting the data representative of the set of identified SVO entity data item(s) may include outputting a graph structure based on the set of identified SVO entity data item(s), the graph structure including a graph of entity nodes with relationship edges linking the entity nodes with each relationship edge including an indication of the directionality of said relationship. Each entity node is associated with an entity of the identified set of SVO entity data items. Each entity relationship is associated with an entity relationship of the identified set of SVO entity data items.

As an option, in step 108, the output data representative of the identified set of SVO data items may include, without limitation, for example, building or updating an SVO search index data structure based on the data representative of the set of SVO entity data items. For example, the graph structure output in step 106 may be used to build or update an SVO search index graph structure associated with one or more domains of interest. The output graph structure may be appended, merged and/or processed for inclusion in the SVO search index graph structure.

As another option, not shown in the FIG. 1a, the output data representative of the identified set of SVO data items may be used as a labelled training dataset including data representative of training labels for use by one or more ML technique for training one or more ML models herein described. The identified set of SVO data items may be represented as a graph structure, which may be used as the training labelled dataset. The one or more ML models may be configured and used to identify, predict and/or extract SVOs or to generalise their prediction thereof.

In step 104, identifying one or more SVO data items may further include identifying meta-data from each of the received text portions for inclusion into each SVO entity data item. The identified meta-data may include data representative of one or more from the group of, without limitation, for example: directionality associated with each entity relationship; biological sign, where applicable, associated with each entity relationship; negation information associated with each entity relationship or affirmation information associated with each entity relationship; context information associated with each entity relationship; any other contextual data associated with said each entity relationship; and any other contextual data associated with the directionality and/or biological sign associated with each entity relationship. In step 106, outputting the SVO data items in the form of a graph structure may include outputting the graph structure in which the relationship edges linking the entity nodes include indications or labels associated with the one or more identified meta-data from the corresponding SVO entity data item(s) associated with the entity nodes. For example, the relationship edge may include an indication of the directionality of the entity relationship between entity nodes.

As described, an entity or entity of interest may comprise or represent an object, item, word or phrase, piece of text, or any portion of information or a fact from a portion of text and the like that may be associated with a particular entity type and be associated with a relationship. Thus, the at least two entities may include data representative of a noun word or a noun phrase associated with the one or more domains of interest. In step 104, the subject entity may correspond to a first noun word or a first noun phrase and the object entity corresponds to a second noun word or a second noun phrase from the one or more domain(s) of interest.

As an example, identifying entities and entity relationship(s) from the one or more text portions may include, without limitation, for example searching the text portions for entities using one or more entity dictionaries or repositories. Each entity dictionary includes a plurality of entities known to be associated with one or more domains of interest. Such known entities may be so-called named entities. Thus, each entity of the at least two entities is a named entity from an entity dictionary associated with at least one of the domain(s) of interest. Additionally or alternatively, in another example, in step 106 identifying, for each portion of text, one or more SVO entity data items may include identifying the first and second entities as named entities from the portion of text based on one or more entity dictionaries associated with said one or more domains of interest. Identifying the first and second entities further includes performing an entity search of the received portions of text based on the one or more entity dictionaries associated with the one or more domain(s) of interest for identifying data representative of at least two entities associated with the one or more domains of interest and an entity dependency relationship therebetween.

The direction or directionality of the relationship, of a particular SVO entity data item, associated with the at least two entities may also be supplemented with the sign or other meta-data associated with the at least two entities. Specifically, meta-data associated with the at least two entities and part of the SVO entity data item, may include but not limited to: an indication of the direction of the identified relationship between said at least two entities based on identified subject and object entities; biological sign, if any, of the identified relationship between said at least two entities based on identified subject and object entities; negation information associated with the identified relationship associated with the between said at least two entities based on identified subject and object entities; context information associated with the identified relationship between the at least two identified entities based on identified subject and object entities; and any other contextual data associated with the relationship between one or more of the at least two identified entities, identified subject entity, identified object entity, verb portion and/or direction.

In one example, when building or updating the SVO search index data structure comprising a set of SVO entity data items, the direction may be away from the subject and towards the object of the SVO triple. The sign may suggest a positive correlation between the subject entity and the object entity of a particular domain of interest, i.e. biological sign between two biological entities. As a result, direction and sign add to the entity dependency relationship indicative of the verb portion between the subject entity and the object entity and may be directed to a syntactic event to which the subject and objects become contextually-linked. Together with the linguistic components (subject entity, verb portion, and object entity) of the SVO triple, the direction and sign effectively strengthen the entity dependency relationship between the subject and the object.

Alternatively or additionally, as previously described, the biological sign may be a label suggesting a positive or negative relationship between said two entities based on identified subject and object entities, where entities may be in, without limitation, for example in the bioinformatics and/or chem(o)informatics domains and the like. For example, the biological sign may be applicable to entity types including at least one or more entity type(s) from the group of, by way of example only but is not limited to, gene, genomics, gene expression and the like; anatomical region or entity; biological pathway, biological process, disease, human disease and the like; antibiotic resistance; compound/drug; protein; tissue; cell; cell-line, or cell type; chemical; organ; food; biological; biomedical; or any other biological or biomedical entity type and the like; or any other entity type of interest associated with the bioinformatics or chem(o)informatics domains and the like. In one example, a pair of entity types associated with the biological sign may be selected from a group of: proteins/genes, diseases, chemicals, mechanisms/processes.

FIG. 1b is a flow diagram illustrating an exemplary SVO identification process 110 for use with or in combination with SVO data item identification step 104 of FIG. 1a according to the invention. The SVO identification process 110 may parse said each received portion of text from the corpus of text for detecting linguistic features associated with the at least two entities associated with the domain(s) of interest and corresponding entity dependency relationship therebetween. The parsing may be accomplished using any number of one or more parsers and/or graph structures for parsing and/or analysing the linguistic features of each text portion for generating each SVO entity data item. The process 110 may identify, from said each received portion of text, a first entity of the at least two entities associated with the subject entity of the received portion of text, a second entity of the at least two entities associated with the object entity of the received portion of text, a verb segment of the entity dependency relationship associated with the verb entity(ies)/portions of the identified relationship in the received portion of text, and other relationship information such as directionality of the relationship between the entities and the like and/or as herein described. The process may output data representative of a set of SVO entity data item(s) representative of the first entity, a segment of the entity dependency relationship, and the second entity. The steps of the process 110 may include the following steps of:

In step 111, the process 110 may perform relationship identification and/or extraction, for each portion of text of a plurality of text portions from the corpus of text, to identify and/or extract at least two entities and relationships thereto from said each portion of text.

For example, step 111 may use, without limitation, for example a relationship identification/extraction ML model that is configured to identify, detect and/or extract entities and/or relationships within each portion of text from the corpus of text. The relationship extraction ML model may be trained based on an ML technique and a labelled training dataset associated with a domain of interest. The labelled training dataset including a plurality of labelled training data items, each labelled training data idem associated with a known one or more entities and an entity relationship thereto. Thus, a selected or specifically designed ML technique may be used with the labelled training dataset to generate a relationship identification/extraction ML model and the like. The relationship identification/extraction ML model may receive a portion of text, process the portion of text, and output data representative of one or more entities and/or a relationship thereto in relation to the portion of text. In effect the relationship identification/extraction ML model searches and/or parses through a plurality of portions of text from the corpus of text to identify, detect and/or extract entities and/or entity relationships and the like.

In another example, step 111 may use, without limitation, for example a rule-based named entity recognition system and one or more entity dictionaries associated with the domains of interest to identify, detect and/or extract entities and/or entity relationships from the portions of text associated with the domains of interest. In effect the rule-based named entity recognition system searches and/or parses through a plurality of portions of text from the corpus of text to identify, detect and/or extract entities and/or entity relationships and the like. Alternatively or additionally, in other examples, step 111 may include using, without limitation, for example one or more named entity recognition system(s) and/or one or more ML model(s) for identifying, detecting and/or extracting entities and/or entity relationships thereto from the plurality of portions of text from the corpus of text corresponding to one or more domains of interest.

In step 112, the SVO identification process 110, for those text portions including identified entities and/or entity relationships, detects linguistic features from one or more segments of said each portion of the text in relation to the identified, detected and/or extracted entities and/or relationships. The process 110 may detect linguistic features from one or more segments of text of the entity relationship that connect the at least two identified entities. The linguistic features may include, without limitation, for example which segments of the text portion is associated with the subject, which segment(s) of the text portion is associated with the object, and which segment(s) of the text portion is associated a verb portion of a relationship between the object and/or subject and the like. This may also include analysing the segment(s) of the text portions associated, without limitation, for example with the verb portions of the relationship to determine the direction of the relationship between the object and/or subject of the text portion. The direction may indicate the relationship is positively or negatively directed to the subject and/or object of the text portion.

In a further example, step 111 may process a text portion from a corpus of text corresponding to, without limitation, for example a biological and chem(o)informatics domain(s) (e.g. disease and chemical/drug). In this example, a text portion includes data representative of “Hydroxychloroquine can not reduce cancer risk in pSS patients”, where a first entity of a chemical entity type that is identified includes “Hydroxychloroquine”, a second entity of the disease entity type that is identified includes “cancer”, and the entity relationship that is identified includes the phrases “[first entity] can not reduce [second entity] risk in pSS patients”. Step 112 may perform linguistic processing and analysis on text portion to identify the linguistic features associated with the first entity, second entity and entity relationship to determine, without limitation, for example an object entity, a subject entity and meta-data associated with enhanced entity relationship information including, without limitation, for example relevant verb portions of the entity relationship, directionality of the identified relationship, negation of the entity relationship, and/or any other further meta-data such as contextual information and the like. Thus, the linguistic processing and analysis determines that the subject-entity is the first entity “Hydroxychloroquine”, the object entity is the second entity “cancer”, and the enhanced relationship information includes the verb portion “reduce”, negation of the entity relationship “can not” or “not” in which the verb portion is found to be “reduce” and directionality may be determined to be from “Hydroxychloroquine” to “cancer”.

In step 113, identifying SVO data representative of the subject entity, object entity, verb portion(s) and indications of direction of the relationship between the subject entity and object entity. In step 113, the process 110 may identify and extract data representative of the subject entity, object entity, verb portions, and direction based on the detected linguistic features from said segments and at least two identified entities and the identified portions of text including identified entities and relationships thereto.

For example, from the text portion, after linguistic processing in step 112, the following text segments may be identified for inclusion into an SVO data item associated with the chemical and disease domains of interest: the subject entity is identified to be the first entity “Hydroxychloroquine”; the object entity is identified to be the second entity “cancer”, and the enhanced relationship information is identified to include a verb portion “not reduce”, which includes the negation of the entity relationship, and an indication of the directionality may be identified to be from “Hydroxychloroquine” to “cancer”, which may be represented by an indicator or flag from a set of directionality indicators or flags that are defined to represent directionality of the entity relationship between the subject entity to the object entity. For example, when the directionality of the relationship is determined to be from the subject-entity to the object entity, then the indicator or flag may be represented, without limitation, for example as a “+” symbol or “→” symbol and when the directionality of the relationship is determined to be from the subject-entity to the object entity, then the indicator or flag may be represented, without limitation, for example as a “−” symbol or “←” symbol, and the like. Although a “+/−” and or “→/←” are described herein to represent directionality, this is by way of example only and the invention is not so limited, it is to be appreciated by the skilled person that any type of indicator, symbol or data representative of a set of directionality indicators/flags may be defined to indicate the directionality of the entity relationship and the like.

Thus, the SVO entity data item for each text portion including entities and entity relationships associated with one or more domains of interest may include: subject entity of the text portion, object entity of the text portion, a verb portion of the entity relationship of the text portion, and meta-data (e.g. directionality, negation, contextual information and the like) associated with the entity relationship of the entities in the text portion. Steps 111 to 113 are performed for each text portion retrieved from the corpus of text, in which a set of SVO data items may be generated and/or built.

In step 114, outputting data representative of a set of SVO data item(s) based on identified SVO data of the plurality of text portions from the corpus of text. The output set of SVO data items may be used in steps 106 and/or 108 of the SVO process 100 for outputting a graph structure and/or building a graph search index structure as described herein. It is apparent that the additional enhanced relationship information associated with each SVO entity data item concisely contains a lot of relevant relationship information of the entity relationship that relates the entities of each SVO entity data item. When the set of SVO data items is represented in a graph structure, this provides an efficient and concise mechanism for enabling researchers and/or other system(s) in a workflow associated with, for example, drug discovery/optimisation and the like, access to the most relevant information associated with entities, entity relationships and the like from the corpus of text (e.g. large scale dataset) within one or more domains of interest. The SVO entity data items may also be provided, in any suitable format, to other system(s), ML model(s), apparatus that may be within a workflow associated with, without limitation, for example, drug discovery and the like.

FIG. 1c is a flow diagram illustrating a further exemplary SVO identification process 115 for use with or in combination with SVO data item identification step 104 of FIG. 1a according to the invention. The SVO identification process 115 may include one or more features and/or steps of the SVO identification process 110 as described with reference to FIG. 1b and the like. The SVO identification process 115 includes the following steps of: In step 116, the process performs relationship extraction for each portion of text to extract at least two entities and relationship. In step 117, detecting linguistic features from the portion of the text, where the portion of the text may be one or more portion of text that connect the at least two identified entities. The portion of text or one or more portion of text that connect the at least two identified entities may be associated with a first entity and a second entity of at least two entities and the entity dependency relationship therebetween. In step 118, extracting subject, verb, and object portion of text associated with the entities and relationship, where there may be at least two entities associated. In step 119, performing meta-data identification such as: negation, sign or biological sign of relationships; the direction or directionality of the entity relationship; and contextual data. In step 120, outputting SVO data item including subject entity, object entity, verb portion, and direction/meta-data.

In operation, the SVO identification process 115 may be configured to determine, for each SVO entity data, at least the sign/entity sign or biological sign and direction of the entity dependency relationship based on a domain mapping engine coupled to an ontological dictionary or any herein described dictionaries of relational terms associated with entities and entity dependency relationships. The dictionaries may be associated with the one or more domains of interest. The domain mapping engine may be configured for determining a segment of the entity dependency relationship representing a sign of the entity dependency relationship for the at least two entities of said each SVO entity data item. For example, sign and direction may be determined via the predefined verb list, from one or more verb lists, that corresponds to the verb portion of the SVO entity item.

In particular, the ontology/dictionary that may be used by the domain mapping engine could originate from a human-made external system, which is used/referred to via, for example, an application program interface (API) or referred to directly from a locally-stored machine readable version. This may be built from a set of training data from a hybrid data, or both, also to include one or more ML model(s) herein described. The ML model(s) may detect terms within the text to be analysed. The training data can originate from human-annotated or labelled data such as SVO labelling or labelling of the associated meta-data for a text portion of an entity pair of interest as described by the figures herein. Alternatively, the ontology/dictionary may be derived from external sources or used in combination with existing dictionaries. Preferably, for a specific domain of interest, the dictionary may be generated in view of that domain of interest.

In one example, when detecting linguistic feature from the portion of the text, ontology/dictionary may comprise a set of biological sign of entity dependency relationship and direction thereof. A particular biological sign may signify a positive relationship in a direction from the first entity to the second entity, where the first entity could be a drug and the second entity a disease. When the domain mapping engine detects a segment within a text or corpus of text that is positively related while having its direction corresponding to that of the ontology/dictionary, for example, a verb such as “treat”, the domain mapping engine may identify and extract the contextual relationship between the first and second entity even if the “treat” was not previously seen by the domain mapping engine.

In another example, ontology/dictionary may comprise a set of SVO labels of previously identified and extract entities of interest, where the dictionary corresponds to a particular domain of interests. Using the dictionary, the domain mapping engine may detect one or more segment of the entity dependency relationship representing a sign of the entity dependency relationship for the at least two entities of said each SVO entity data item. From the segment the SVO entity data item may be identified and extracted in relation to the particular domain of interest, which includes but not limited to biology, biochemistry, chemistry, medicine, chem(o)informatics, bioinformatics, pharmacology, and/or any other field relevant to diagnostic, treatment, and/or drug discovery and the like.

In a further example, additionally or alternatively, the domain mapping engine may be implemented or configured by looking up any portion of the SVO data items or combination of them including, without limitation, for example the context, subject, object entities, relationship information and/or verb portion and the like of an SVO entity data item using predefined tables of contexts, relationships, verbs and the like that describe affirmation information, negation information, positive sign, negative sign, directional, and non-directional. Alternatively and additionally, the domain mapping engine may adapt to the use of one or more ML model(s) herein described and configured to identify, predict and/or extract affirmation, negation, sign and/or direction information, where the one or more ML model(s) may be automatically/semi-automatically trained by one or more ML technique(s) using labelled training datasets such as, without limitation, for example annotated sentences and/or text portions and the like.

FIG. 1d is a flow diagram illustrating another exemplary SVO identification process 130 for use with or in combination with SVO data item identification steps 104 of FIG. 1a according to the invention. In addition to the SVO identification process 130 described below, the SVO identification process 130 may be further modified and/or combined with one or more features and/or steps of the SVO identification process(es) 110 and/or 115 as described with reference to FIGS. 1b and/or 1c and the like. As illustrated in FIGS. 1d, in step 131 portions of text from the corpus of text or segments thereof as textual data may be received. In step 132, detecting entities of interest or entities associated with one or more domains of interest based on the received textual data (or portions of text), which may be processed to detect statement(s) containing entities of interest for use in identifying SVO entity data. In step 133, extracting the entities of interest from the detected textual data or detected portions of text as related pair of entities. For example, text segments or words/phrases associated with a first entity, a second entity and/or entity relationship therebetween may be extracted. In step 134, each textual data or each text portion including the extracted entities may be further processed for detecting linguistic features connecting the entities may be detected 134. Once linguistic features are detected, the SVO identification process 130 may, in step 135, extract subject, verb, object, meta-data and/or contextual information from the identified linguistic features for each textual data or text portion. The meta-data and/or contextual information includes, without limitation, for example data representative of the associated negation information, directionality information, and, if any, biological sign and the like. The process 130 may, in step 136, determine and/or create directional relationship between the entities based on the entity relationship therebetween. This may include negation, context, and perhaps other extracted contextual information. The SVO identification process 130 identifies and extracts the meta-data such as negation information, which also includes but is not limited to, signed and directional relationship from textual data via identifying a pair of entities.

In particular, the SVO identification process 130 identifies and extracts SVO information associated with an entity pair and relationship therebetween to form SVO data item including data representative of the identified object entity, the identified subject entity, an identified verb portion and associated meta-data that may include but not limited to negation information, sign/biological sign, indication of the direction, context information, and any other textual data associated with the entity relationship between one or more of the at least two identified entities, identified subject entity, identified object entity, verb portion and/or direction. The SVO entity data item(s), which includes extracted meta-data, may be forwarded to other systems or processes of the present invention, distinctively or in the form of an SVO data item. The identification and extraction of the meta-data via literature-based evidence that is associated with one or more domain(s) of interest of relationship, between a pair of entities, the SVO identification process 130 produces syntactic events. These syntactic events or linguistic features may be used to strengthen the relationship between a pair of entities, for example, utilising the syntactic event or occurrence with respect to direction, negation, and context and/or other extracted information as described in step 136 of FIG. 1d.

In one example, a text portion such as a statement is identified that contains a pair of entities of interest from steps 131, 132, and 133 of FIG. 1d. The statement contains potential relationships between the pair of entities, without limitation, for example biomedical entities from the biomedical domain and/or biological/chem(o)informatics domain(s). For example, the biomedical entities may be a drug and a disease. The relationship between the drug and the disease may be determined based on the linguistic detection in step 134 to be connected by a verb word/phrase, such as, without limitation, for example, “reduce”, which links the drug to disease. This also concisely describes the entity relationship between the drug and the disease. Although the term “reduce” is used, this is by way of example only and the invention is not so limited, it is to be appreciated by the skilled person that any other suitable verb word/phrase and/or term concisely describing the entity relationship may be derived from the linguistic detection of the verb portion of the entity relationship between entities. The terms connecting the drug and the disease are also further analysed linguistically in step 134 of the FIG. 1d so that the Subject entity, Verb entity, Object entity (or SVO type information) may be detected and then extracted in step 135, the detected SVO type information may be used to add further meta-data and/or relationship information, without limitation, for example sign and direction to the entity relationship between these biomedical entities as shown in step 136. Similarly, additional relationship information or meta-data may be detected during the linguistic analysis of step 134 and extracted in step 135 associated with the context of the statement that can add further concise details of the extracted entity relationship between the entities.

FIG. 1e is a flow diagram illustrating yet another exemplary SVO identification process 140 for use with or in combination with SVO data item identification step 104 of FIG. 1a according to the invention. In addition to the SVO identification process 140 described below, the SVO identification process 140 may be further modified and/or combined with one or more features and/or steps of the SVO identification process(es) 110, 115 and/or 130 as described with reference to FIGS. 1b, 1c and/or 1d and the like. The SVO identification process 140 may include one or more of the following steps of: In step 142, the SVO identification process 140 receives entity results including entity pairs and entity relationship(s) therebetween. This may be in the form of data representative of a list of entity pairs in which each entity pair has an entity relationship therebetween. In step 144, the process identifies SVO entity data item(s) in the form of SVO triple(s) from each of the entity pairs of the entity results. In step 146, the process determines meta-data such as, without limitation, for example sign and direction data (including other context information) of the entity relationship between entity pairs for each SVO triple. In step 148, the process 140 outputs and/or stores a set of one or more SVO triple(s) and corresponding signed/direction and/or associated meta-data. For example, the set of SVO triple(s) may be output in the form of a graph structure and/or stored in a search graph index structure and the like and/or as described herein with reference to FIGS. 1a-7b.

For example, an SVO triple may include data representative of a subject, a verb portion, and an object associated with a pair of entities and the entity relationship therebetween and further includes the extracted meta-data (e.g. sign/direction/context). The subject of one of the SVO triples is associated with a first entity of the at least two entities, the object of said one of the SVO triples is associated with a second entity of the at least two entities, and the verb of said one of the SVO triples is associated with the entity dependency relationship between the first and second entities. This in effect forms the SVO triple. Alternatively, the SVO triple may be formed where the object of one of the SVO triples is associated with a first entity of the at least two entities, the subject of said one of the SVO triples is associated with a second entity of the at least two entities, and the verb of said one of the SVO triples is associated with the entity dependency relationship between the first and second entities.

In one example, the process and system may generate or update a knowledge graph structure based on the output and/or stored set of SVO triples. An SVO triple may be in the form of two entities and an entity relationship (also called an entity dependency relationship). The set of SVO triples may be identified by at least two entities and their entity relationship(s).

The subject of one of the SVO triples may be associated with a first entity of the at least two entities. The object of said one of the SVO triples may be associated with a second entity of the at least two entities. The verb of said one of the SVO triples may be associated with the entity relationship between the first and second entities. For each identified SVO triple, meta-data representative of at least the direction of the entity relationship between the first and second entities corresponding to said each SVO triple is determined. An SVO entity data item may include data representative of an identified SVO triple and the associated meta-data including, without limitation, for example at least the direction of the entity relationship between the first and second entities of said identified SVO triple. Thus a set of SVO entity data item(s) may be generated/created from the entity results using process 140 and output to generate or update the knowledge graph structure. The possible structures of the knowledge graph may include, by way of example only but is not limited to, directed, undirected, vertex labelled, cyclic, edged labelled, weighted, and disconnected graph or subgraphs and the like, and/or any other suitable graph structure for concisely and efficiently representing the identified SVO triples and meta-data corresponding to the set of SVO data items. Various algorithms may be used to traverse or search the graphs for extracting subsets of graphs and/or subgraphs based on search queries associated with the entities, concepts and/or entities within the domains of interest and the like.

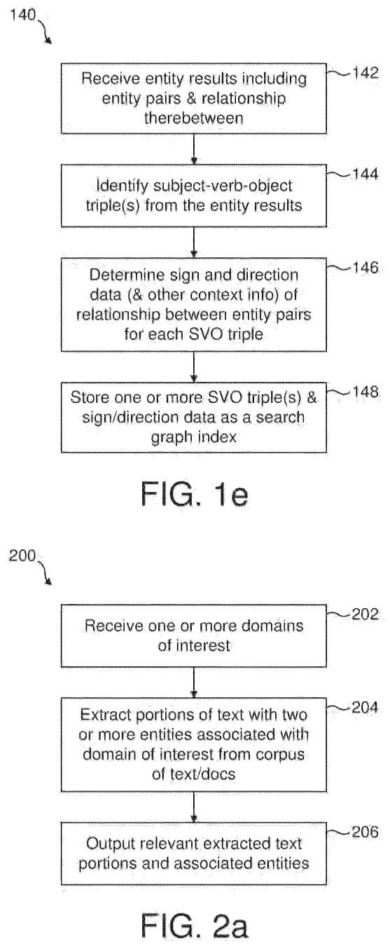

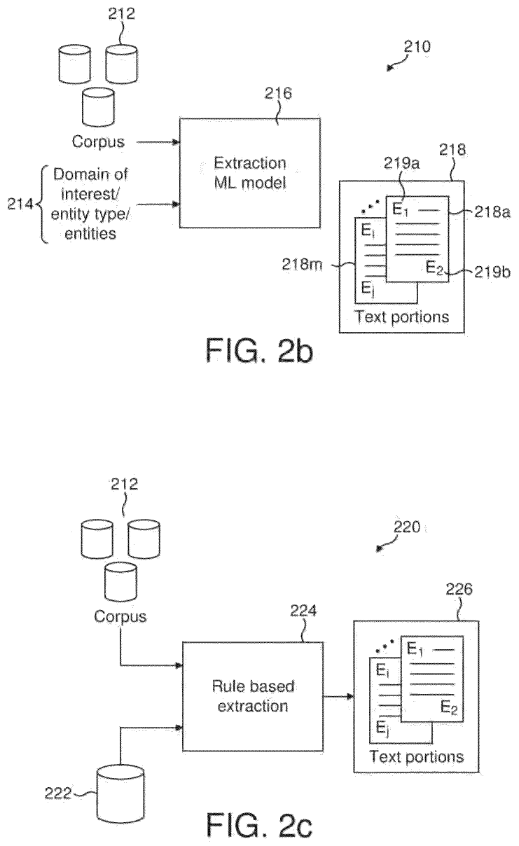

FIG. 2a is a flow diagram illustrating an exemplary process 200 for extracting portions of text from a corpus of text associated with one or more domains of interest, in which the portions of text may include one or more entities and/or associated relationships for use by the SVO identification processes 100, 115, 130, 140 as described with reference to FIGS. 1a to 1 e according to the invention. In step 202, the process 200 receives a request for retrieving candidate text portions associated with one or more domains of interest. In step 204, the process 200 identifies suitable corpus of text associated with the one or more domains of interest and extracts candidate portions of text with two or more entities associated with the domain of interest from the corpus of text/documents. This may include using ML model(s) configured for identifying candidate portions of text with entities from said one or more domains of interest. This may include using rule based systems and entity dictionaries or named entity recognition systems associated with the one or more domains of interest for identifying candidate portions of text from the corpus of text. Additionally or alternatively, this may include combined ML model(s) and/or rule based systems and/or entity dictionaries associated with the one or more domains of interest and configured for identifying text portions from the corpus of text. In step 206, the process 200 outputs relevant extracted text portions including entities associated with the one or more domains of interest. One example of the domain of interest may be biology in nature, to which a biological sign of the entity dependency relationship and data representative of the direction indication of the entity dependency relationship may be derived. Specific examples of the biological sign may include but not limited to “positive” and “directed” as well as to quantitative value between 0 and 1, where 1 suggests “positive” and 0 suggests “not positive”.

FIG. 2b is a schematic diagram illustrating an ML model text extraction system 210 based on the process of FIG. 2a using ML techniques according to the invention. As illustrated, a text portion extraction ML model 216 is configured to identify, detect and/or extract portions of text 218 (e.g. paragraphs, statements, sentences, phrases, and the like) including at least two entities (219a/b) associated with the one or more domains of interest 214, entities/entity types and the like and an entity relationship therebetween from a corpus of text or documents 212. In particular, the extraction ML model 216 receives text, documents, data and/or portions of text from the corpus of text 212 associated with the one or more domain(s) of interest 214, entities/entity types and the like. The extraction ML model or model(s) 216 may be configured to identify or predict entities (e.g. E1 219a or E2 219b) associated with the one or more domains of interest 214 and/or entity relationships associated with the one or more domains of interest and output candidate text portions 218a to 218m including one or more entities (e.g. E1 219a or E2 219b) and entity relationships therebetween. Any of the herein described ML technique(s) may be used or be part of the text portion extraction ML model 216 for the purpose of identification and prediction. The extracted portions of text 218a to 218m including entities associated with one or more domains of interest 214 and/or relationships thereto may be input to the SVO process(es) as described with reference to FIGS. 1a to 1e and the like for identifying SVO entity data items from the extracted portions of text 218a-218m.

Similarly as described for FIG. 2b, FIG. 2c is a schematic diagram illustrating an entity extraction system 220 based on the process of FIG. 2a using rule-based and entity dictionary techniques for extracting portions of text from a corpus of text associated with one or more domains of interest according to the invention. The text extraction system 220 includes a rule-based text extraction engine 224 that is configured to identify and/or extract candidate portions of text from the corpus of text 212 associated with the one or more domains of interest and output candidate text portions including one or more entity(ies) associated with the one or more domains of interest based on an entity search or rule-based NER system(s) and the like, using the entity dictionaries 222 associated with the one or more domains of interest, of the text, data and/or portions of text from the corpus of text 212. The system 220 extracts candidate text portions 226 from the corpus of text, each candidate text portion including one or more entities associated with the one or more domains of interest and an entity relationship associated with the one or more entities. For example, a candidate text portion may include data representative of at least two entities associated with the one or more domains of interest and an entity dependency relationship therebetween. The extracted portions of text 226 including entities associated with one or more domains of interest and/or relationships thereto may be input and/or used by the SVO process(es) as described with reference to FIGS. 1a to 1e and the like for identifying SVO entity data items from the extracted portions of text.

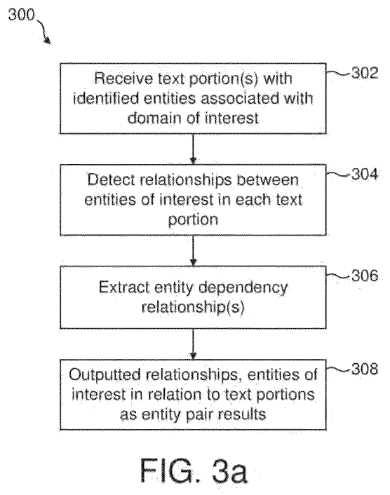

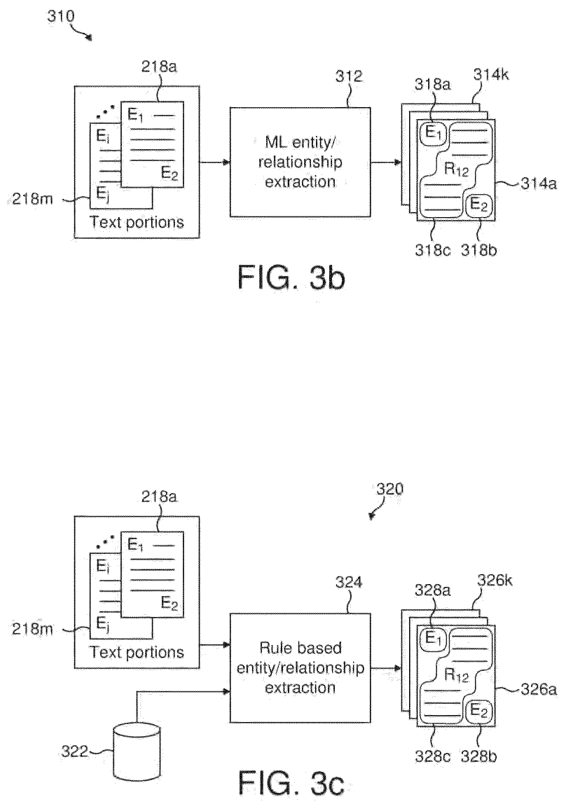

FIG. 3a is a flow diagram illustrating an exemplary process 300 for extracting entities and/or entity relationships from text portions including entity pairs for use by the SVO process(es) of FIGS. 1a to 1e according to the invention. In step 302, the process 300 receives a plurality of text portions, where each received text portion includes data representative of entities associated with the domain(s) of interest. The text portion may include identified entities associated with the domain of interest, such as a text portion output from process(es) 200 and/or systems 210 and/or 220 and the like. In step 304, the process 300 detects the entity relationship between the entities of interest identified in each text portion. For example, this may be achieved using ML relationship extraction models and/or rule based systems configured for detecting and/or identifying entities associated with the domains of interest, and then extracting the text segments corresponding to the entity relationship associated with the identified entities. Alternatively or additionally, the rule-based systems may use one or more entity dictionaries, one or more entity relationship dictionaries and the like for use in identifying the text segments of the text portion associated with an entity relationship in relation to the identified entities and the like. A relationship dictionary, database, and/or storage may be configured to store data representative of one or more entity relationships, where an entity relationship includes data representative of, without limitation, for example one or more text segment(s), text portion(s), string of character(s), phrases, words, sentences, verb portions and the like that corresponds or constitutes a relationship associated with one or more entity(ies). Relationship dictionary may be used to store and/or retrieve entity relationships and the like for use with the process(es)/method(s) herein described. In step 306, the process may extract the identified entity dependency relationship(s) and/or the identified entities associated with the identified entity relationship(s). In step 308, the process outputs the entity relationships, entities associated with the domains of interest in relation to the received text portion(s). This may be in the form of data representative of entity results, which include data representative of a set of one or more identified entities and relationships thereto. For example, the entity results may be output as, without limitation, for example data representative of a set of entity pairs, in which each entity pair is associated with an entity relationship thereto. In another example, the entity results may be in the form of a set of entities with associated relationship information. Alternatively or additionally, the entity results may be output as a graph structure in which each entity corresponds to an entity node, and each relationship is associated with an edge linking to one or more entities within the graph structure to other entities within the graph structure. This may be processed and used in the SVO process(es) as described with reference to FIGS. 1a to 1e and the like.

In one example, without limitation, for example the entity results may be sets of biological entity pairs from a domain of interest related to biological sciences. Entity results may further include at least two or more entities of interest or data representative thereof, which include but not limited to an entity type in relation to a domain of interest from a subgroup such as, by way of example only but not limited to, bioinformatics and/or chem(o)informatics, and/or any other domain of interest and the like. The subgroup may be used in relation to one or more alternative domains of interest such as chem(o)informatics, data informatics, social media, and entertainment, geographical, any other entity type in which a portion of text comprises data representative of a relationship for one or more entity(ies).

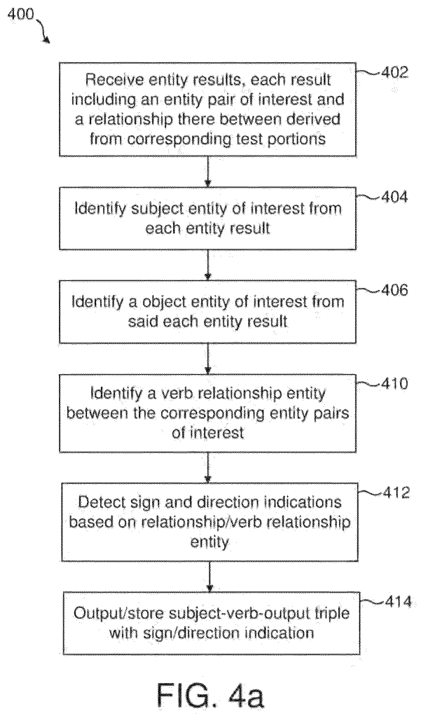

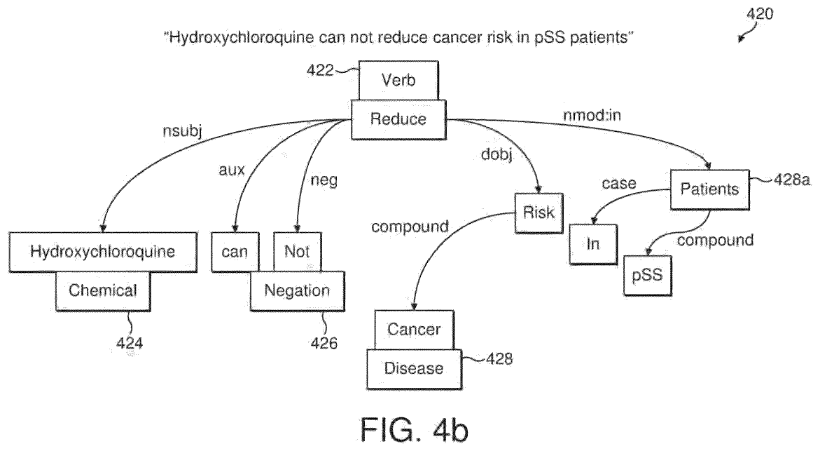

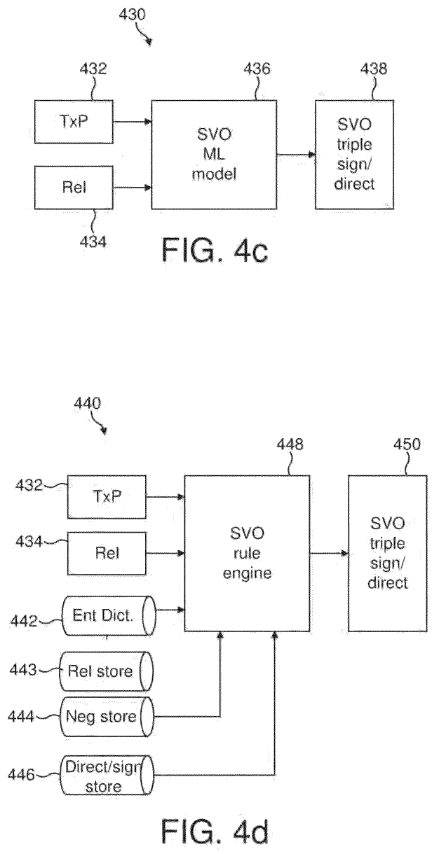

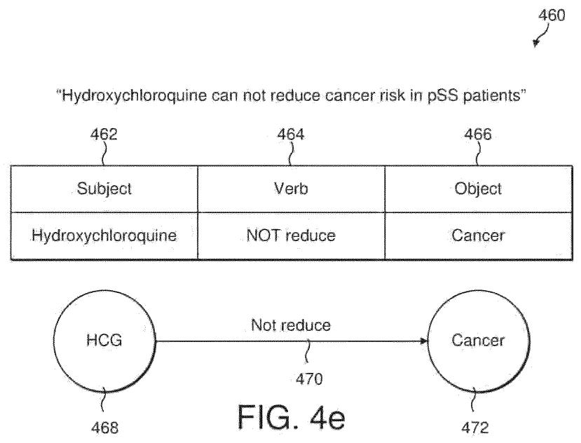

FIG. 3b is a schematic diagram illustrating an entity relationship extraction system 310 based on the process of FIG. 3a using machine learning (ML) techniques according to the invention. For simplicity, reference numerals for similar or the same features used with reference to FIGS. 2a to 2c are re-used. The entity relationship extraction system 310 may take as input, without limitation for example, extracted portions of text 218a-218m or 226 that may be output from extraction ML model 214 and/or rule-based extraction 224 as described with reference to FIG. 2b or 2c. As illustrated, the entity relationship extraction system 310 includes one or more ML relationship extraction model(s) 314 that are configured to receive the portions of text 218a to 218m, which may be extracted and/or found from a corpus of text associated with the one or more domain(s) of interest as described, by way of example only but not limited to, with reference to FIGS. 2a to 2c. Each of the portions of text may include identified entities associated with one or more domains of interest as described with reference to FIGS. 2a to 2c. Using the one or more ML relationship extraction (RE) model(s), the text portions 218a-218m from the corpus of text associated with one or more domains of interest are input to the ML RE model(s), which have been trained to identify, detect and/or extract one or more entities of interest and/or relationships thereto, where the portions of text 218a to 218m are processed to identify entities and relationships thereto. For example, a portion of text 218a may be processed by ML RE model 312 to output data representative of entity result 314a that includes at least two entities 318a and 318b in one or more domain(s) of interest and an entity dependency relationship 318c associated therewith. The ML RE model(s) may output a set of entity results 314a-314m corresponding to those processed text portions 218a-218m that had identified entities 318a and 318b and relationships 318c. Each entity result 314a includes data representative of one or more entities 318a-318b associated with the domain(s) of interest and entity relationship 318c associated with the one or more entities (e.g. E1, E2). The entity results 314a-314k may be input to the linguistic process(es) as described with reference to FIGS. 1a to 1e and/or FIGS. 4a-7b and/or as herein described for use in determining SVO data item(s) including an object entity, subject entity, verb portion and meta-data including, without limitation, for example biological sign of the entity relationship, directionality associated with the entity relationship, negation sign associated with the entity relationship and/or any other contextual/meta-data for enhancing the entity relationship information that may be extracted and the like.

FIG. 3c is a schematic diagram illustrating another entity relationship extraction system 320 based on the process of FIG. 3a using a rule-based and relationship dictionary techniques according to the invention. For simplicity, reference numerals for similar or the same features used with reference to FIGS. 2a to 2c are re-used. The entity relationship extraction system 320 may take as input, without limitation for example, extracted portions of text 218a-218m or 226 that may be output from extraction ML model 214 and/or rule-based extraction 224 as described with reference to FIG. 2b or 2c. As illustrated, the entity relationship extraction system 320 includes a rule-based relationship extraction process/apparatus 324 (e.g. rule-based NER system and the like) that is configured to receive portions of text 218a-218m or 226 output from the ML extraction model 214 and/or rule-based extraction system 224 (e.g. sentences, paragraphs, statements, documents, text segments and the like) that were found from the corpus of text associated with the one or more domain(s) of interest. A relationship server, relationship/entity dictionary(ies) 322 associated with the domain of interest may be used for assisting the rule-based entity/relationship extraction system 324 in identifying, detecting and/or extracting one or more entities and relationships thereto. Each portion of text 218a-218m (or 226) may include data representative of one or more entity(ies) and/or data representative of an entity relationship associated with the one or more entity(ies). Each portion of text may be based on the candidate portions of text extracted using process 200 and/or systems 220 and 230 as described with reference to FIGS. 2a to 2c. Using rule based relationships extraction process/apparatus 324, entities and/or entity relationships thereto are identified from the received portions of text 218a-218m. The rule-based relationships extraction process/apparatus 324 may take as input one or more exemplar relationships and/or entities from a relationship store/entity dictionary(ies) and the like 322 associated with the domains of interest. The processed text portions 326a-326k including one or more entity(ies) 328a-328b associated with the one or more domains of interest may be identified based on an entity search of the received portions of text 218a-218m using on one or more entity dictionaries, and/or performs a entity relationship search using the one or more relationship stores/entity dictionary(ies) and the like 322 associated with the one or more domains of interest.

Alternatively, the rule-based relationship extraction system 324 may identify the entities in each portion of text and then determine which segment of text associated with the identified entities is associated with the entity relationship. The rule-based entity relationship extraction system 324 identifies and/or extracts, from each identified text portion 326a-326k one or more entities 328a-328b and/or an entity relationship 328c corresponding with data representative of at least two entities (e.g. E1 and E2) associated with the one or more domains of interest. The text portions 326a-326k may represent a set of entity results 326a-326k, each entity result 326a including one or more entities 328a-328b and an entity relationship thereto 328c. The set of entity results 326a-326k may be input to the linguistic process(es) as described with reference to FIGS. 1a to 1e and/or FIGS. 4a-7b and/or as herein described for use in determining SVO data item(s) including an object entity, subject entity, verb portion and meta-data including, without limitation, for example biological sign of the entity relationship, directionality associated with the entity relationship, negation sign associated with the entity relationship and/or any other contextual/meta-data for enhancing the entity relationship information that may be extracted and the like.

FIG. 4a is a flow diagram illustrating an exemplary SVO process 400 for generating subject-verb-object triples from entity pairs and associated entity dependency relationships according to the invention. In step 402, the SVO process receives entity results, each entity result including an entity pair of interest and a relationship therebetween derived from corresponding text portions. The entity results may be received based on the process(es) and/or system(s) as described with reference to FIGS. 2a to 3c. In step 404, the SVO process 400 uses linguistic analysis techniques and the like to identify the subject entity of interest from each entity result. In step 406, the SVO process 400 uses linguistic analysis techniques to identify an object entity of interest from said each entity result. In step 410, the SVO process 400 uses linguistic analysis techniques to identify a verb portion of the entity relationship between the corresponding entity pairs of interest. In step 412, the SVO process 400 also uses linguistic analysis techniques to detect data representative of, without limitation, for example sign and/or direction indications of the verb portions and/or entity relationship of said each entity result. In step 414, the SVO process 400 outputs and/or stores a set of SVO entity data items including an SVO triple with meta-data including data representative of the sign and/or direction indication as described with reference to FIGS. 1a to 3c and/or with reference to FIGS. 4b to 7c and/or as described herein.

FIG. 4b is a schematic diagram illustrating an example SVO linguistic data structure 420 based on performing a linguistic analysis of each text portion including one or more entities and an entity relationship as described with reference to SVO process 400 of FIG. 4a according to the invention. As illustrated in FIG. 4b, a received text portion including at least two entities associated with one or more domains of interest (e.g. biological and disease domains) is analysed using linguistic analysis techniques to build an SVO linguistic data structure 420 in the form of a vocabulary graph structure. The SVO process 400 performs SVO identification using linguistic analysis techniques on the received text portions to identify, without limitation, for example a subject entity corresponding to an entity of the at least two identified entities, an object entity corresponding to an entity of the at least two identified entities, and a verb portion associated with the identified entity relationship, and/or further meta-data including, without limitation, for example, affirmation or negation information associated with the identified entity relationship and/or indication of direction of the entity relationship. In particular, SVO process 400 detects the linguistic features from each of the received portions of text from a plurality of text portions, and for each text portion, identifies the words/phrases associated with the at least two identified entities, identifies and analyses the entity relationship and extracts relevant information associated with the relationship including verb portions, negation portions, directionality indications and the like and/or any other contextual data associated with the entity relationship, and extracts data representative of the subject entity, object entity, verb portions, negation and/or direction based on the at least two identified entities, and adds the extracted direction indication to the relationship associated with the at least two entities.

As illustrated in FIG. 4b, the following example text portion or statement is received: “Hydroxychloroquine can not reduce cancer risk in pSS patients”, which is analysed by process 400 using linguistic techniques that identify the linguistic role of each segment, word and/or phrase within the received text portion or statement. In this example, the identified linguistic features include, without limitation, for example Subject (nsubj), Verb, Object (dobj) can be used to determine the relationship type and/or direction. Using these linguistic features, an SVO linguistic data structure 420 may be formed and processed for determining and extracting the subject entity, object entity, verb portions, and/or meta-data associated with negation, direction of the relationship, any biological sign associated with the relationship and any further contextual data further describing the relationship. In this example, the statement “Hydroxychloroquine can not reduce cancer risk in pSS patients” is processed which may identify a first entity as “Hydroxychloroquine”, a type of chemical (e.g. from the chemical domain) 424 is related to a second entity “Cancer” 428, a type of disease (e.g. from the disease domain) and that the entity relationship includes the words/phrases “can not reduce [second entity] risk in pSS patients”. From this, the SVO linguistic data structure 420 may be formed for determining the enhanced relationship information or meta-data associated with the entity relationship such as, without limitation, for example the direction of the entity relationship being from Hydroxychloroquine to Cancer, and with the entity relationship being a negative 426 “not” with regards to the term “not reduce” in relation to the verb portion “reduce” 422. In terms of generating the SVO data item for this example text portion, the subject entity is identified to be “Hydroxychloroquine”424, the Verb portion is processed and identified to be “Not Reduce” 422/426 and the object entity is identified to be “Cancer” 428.

Additional terms or information 428a from the entity relationship may be used as additional meta-data such as, without limitation, for example contextual data, which are shown in grey and may add further meta-data and/or relevant relationship information for the entity relationship. The additional term or relationship information may include, for example but is not limited to, disease, cases, compounds/auxiliary terms, and the like. These terms add meta-data such as the anatomical location for the entity relationship, whether the entity relationship is associated with the organ, tissue, etc., patient and the like. This example of the SVO process 400 and SVO linguistic data structure 420 for identifying, labelling segments of a text portion or statement of an entity pair of interest, in effect, and extracting the relevant labelled segments may be used to form an SVO entity data item. This may be repeated for a plurality of text portions from a corpus of text for generating a set of SVO data items, which may be output as data representative of a graph structure as described herein with reference to FIGS. 1a to 4a and 4c to 7c and the like, where nodes are entities from the set of SVO entity data items and the links between them could embed relationship meta-data from the set of SVO entity data items such as indications of sign and direction.

For example, the graph structure may be used to build a SVO search index data structure or graph structure, which may be queried using one or more search queries associated with entities from one or more domains of interest used to generate the graph structure. In turn, the SVO entity data items may be stored in, without limitation, for example a relational database or other storage media and may be in the form of data representative, without limitation, for example, a knowledge graph, where nodes are entities and the links between them could embed meta-data such as being both signed and directional.

In another example, inferred entity dependency relationships may be derived from a statement(s) such as “Infection of DCs with live Mtb led to cell death.”, where “Infection” is the subject entity, “Led to” is a Verb portion of the entity relationship, and the object entity is “Cell Death”. In addition, the relationship between “DCs with live Mtb” may be correlated with both “infection” and “cell death” such that alterative entity dependency may be inferred such as between ‘DCs” and “cell death”. In a further example, in another statement, “GPR-9-6 was expressed at high levels in thymus.”, where the identified subject entity is “GPR-9-6”, the Verb portion of the entity relationship is identified to be “Expressed in”, and the identified object entity is identified to be “Thymus”. In this example, the phrase in the entity relationship “expressed at high levels” may be identified to indicate a positive relationship or positively in the direction from the object entity to the subject entity.

The SVO process(es) as described herein differs from a simple co-occurrence type relationship extraction model in that, the SVO process(es) use meta-data as the means to extract from each received portion of text concise, meaningful and more accurate relationship data from the entity relationship that can be used for more accurately deriving any inferences between entity nodes of a graph structure thereof. The set of SVO entity items produced as the result of the SVO process(es) as described herein includes not only the fact that the subject entity is related to the object entity, but also the way in which it is related using verb portions and deduced from meta-data such as direction and/or sign and the like. This produces an unparalleled advantage with respect of the amount of relevant relationship information that may be automatically derived and/or processed from a large-scale corpus of text including text portions and output into an efficient and concise data structure such as, without limitation, for example a graph data structure, that may be parsed, searched as a search index and/or displayed to users rather than provision of an overwhelming multitude of tabulated entity results output by conventional systems.

FIG. 4c is a schematic diagram illustrating an SVO linguistic system 430 for use with the process of FIG. 4a using machine learning (ML) technique(s) according to the invention. As illustrated, the SVO linguistic system includes an SVO ML model 436 receives a plurality of text portions, each text portion 432 (e.g. TxP) including one or more identified entities and corresponding entity relationship(s) 434 (e.g. Rel) for input to the SVO ML model 436. In this example, text portions from the corpus of text may be processed to identify the entities and entity relationships based on the process(es) and/or system(s) as described with reference to FIGS. 2a to 3c, and/or as herein described with reference to FIGS. 1a to 7c and the like. The SVO ML model 436 may be generated based on one or more ML technique(s) that operate on a labelled SVO training dataset for training the SVO ML model 436. The labelled SVO training dataset may be based on known text portions labelled or annotated with entities and relationships and corresponding SVO linguistic features. The SVO labelled training dataset may include a plurality of labelled text portion data items, with each labelled text portion data item having segments or words labelled with corresponding SVO linguistic features such as, without limitation, for example an object entity, a subject entity, a verb portion and/or meta-data including, without limitation, for example, indication of direction of the entity relationship and/or negation and other contextual information. For example, the SVO ML model 436 may be trained and configured for, on receiving a text portion from a corpus of text, identifying and/or extracting SVO linguistic features such as, without limitation, for example an object entity, a subject entity, a verb portion and/or meta-data including, without limitation, for example, indication of direction of the entity relationship and/or negation and other contextual information.

The SVO ML model may be configured to, on receiving a text portion 432 from a corpus of text, identify and extract the required SVO data for forming an SVO entity data item for said each text portion 432 and the like. For example, the SVO ML model may be configured to identify, extract and output a subject entity, an object entity, a verb portion, and/or meta-data associated with the entity relationship and the like based on an input text portion 432 and/or segments thereof. The SVO ML model 436 may separately receive segments of each text portion 432 in which a first segment includes data representative of the one or more identified entities and a second segment including data representative of the corresponding entity relationship(s) 434. Alternatively, the SVO ML model 436 may simply receive data representative of the text portions 432, with each text portion including data identifying the one or more entities and corresponding entity relationships within each text portion. In this example, a text portion 432 is depicted as a text portion including data representative of entities and a text portion including data representative of the corresponding entity relationship 434 in relation to the entities. The SVO ML model 436 identifies, for each text portion 432 or segments of a text portion that are input, one or more SVO linguistic features and may output data representative of an SVO entity data item 438. In this example, the SVO ML model 436 may output an SVO entity data item 438 including data representative of an SVO triple, which includes a subject entity, a verb portion associated with the entity relationship, an object entity, and/or meta-data such as, without limitation, for example sign and/or direction indication of the entity relationship and the like. Thus, should a set of text portions 432 be input to the SVO ML model 436, these may be processed in which the SVO ML model 436 outputs corresponding set of SVO data items 438.

FIG. 4d is a schematic diagram illustrating another example SVO linguistic system 440 for use in the linguistic techniques associated with identifying SVO linguistic features as described with reference to SVO process 400 of FIG. 4a. The SVO linguistic system 440 may be a rule-based and/or dictionary based system that parses each received text portion 432 and/or corresponding relationship 434, identifies and/or extracts SVO linguistic features for outputting an SVO entity data item associated with the received text portion 432 and/or corresponding relationship 434. The SVO linguistic system 440 may include one or more entity dictionaries or stores 442 (e.g. Ent. Dict.), relationship dictionaries or stores 443 (e.g. Rel store), negation dictionaries/stores 444 (e.g. Neg store) (or affirmation dictionaries/stores (not shown)), direction/sign dictionaries/stores 446 (e.g. Direct/sign store) that are coupled to an SVO linguistic rule-based engine 448 configured for parsing, identifying and/or extracting SVO linguistic features from each received text portion from the corpus of text. A relationship dictionary, database, and/or storage may be configured to store data representative of one or more entity relationships, where an entity relationship includes data representative of, without limitation, for example one or more text segment(s), text portion(s), string of character(s), phrases, words, sentences, verb portions and the like that corresponds or constitutes a relationship associated with one or more entity(ies). Relationship dictionary may be used to store and/or retrieve entity relationships and the like for use with the process(es)/method(s) herein described. The rule-based system 440 and/or SVO linguistic rule-based engine 448 uses a set of rules for parsing, identifying and/or extracting SVO linguistic features using one or more of the entity dictionaries or stores 442 (e.g. Ent. Dict.), relationship dictionaries or stores 443 (e.g. Rel store), negation dictionaries/stores 444 (e.g. Neg store), direction/sign dictionaries/stores 446. A rule-based system may extract SVO data items based on the following manner: When the rule-based system is provided with a sentence or a text portion 432, a dependency graph may be produced by a third-party natural language processing framework (e.g. Stanford CoreNLP or Spacy) that corresponds to the sentence or text portion 432. The text portion 432 and dependency graph may be processed by the rule-based system 440 in which the shortest “dependency path” (DP) on the dependency graph between the each pair of entities E1 and E2 in the text portion 432 (e.g. E1 could be a disease and E2 a gene) is extracted. Then a first set of rules based on the dependency labels and the part of speech tags allow the rule-based system 440 or SVO rule engine 448 to decide whether this DP corresponds to a Subject-Verb-Object pattern and identifies which terms along the path correspond to the verb. Further detection of context entities may be accomplished using a second set of rules to check/verify which entities in the sentence or text portion 432 are related to the subject, the verb or the object.

Each text portion 432 includes one or more entity(ies) associated with the one or more domains of interest and an entity relationship thereto. In this example, text portions 432 from the corpus of text may be processed to identify the entities and entity relationships based on the process(es) and/or system(s) as described with reference to FIGS. 2a to 3c, and/or as herein described with reference to FIGS. 1a to 7c and the like. The SVO ML model 436 may separately receive segments of each text portion 432 in which a first segment includes data representative of the one or more identified entities and a second segment including data representative of the corresponding entity relationship(s) 434. Alternatively, the SVO ML model 436 may simply receive data representative of the text portions 432, with each text portion 432 including data identifying the one or more entities and corresponding entity relationships within each text portion. In this example, a text portion is depicted as a text portion 432 including data representative of entities and a text portion including data representative of the corresponding entity relationship 434 in relation to the entities.

The one or more entity dictionaries or stores 442, relationship dictionaries or stores 443, negation dictionaries or stores 444 and/or direction/sign stores or dictionaries 446 are associated with the one or more domains of interest. The SVO linguistic rule-based engine 448 extracts, from each identified text portion, data representative of one or more entities (e.g. at least two entities) associated with the one or more domains of interest and an entity relationship therewith. The SVO rule-based engine 448 identifies SVO linguistic features for forming, for each text portion, an SVO entity data item 450. The SVO entity data item 450 may be in the form of an SVO triple based on the one or more entities (e.g. at least two entities) and an entity relationship therewith. The SVO entity data item 450 may include data representative of a subject entity corresponding to a first entity of, for example, at least two entities, an object entity corresponding to a second entity of the at least two entities, and the verb portion associated with the entity relationship in relation to the first and second entities. The SVO entity data item 450 may also include meta-data representative of at least the sign and/or direction of the entity relationship between the first and second entities. Thus, should a set of text portions be input to the SVO linguistic rule-based engine 448, these may be processed in which the SVO linguistic rule-based engine 448 outputs a corresponding set of SVO data items.

Although an SVO ML model and/or SVO linguistic rule-based engine 448 are described herein, this is by way of example only and the invention is not so limited, it is to be appreciated by the skilled person that a combination of SVO ML model(s) and/or SVO linguistic rule-based engine(s) 448 may be used, modifications thereof, and/or any other type of linguistic technique and/or natural language processing (NLP) techniques may be used and/or performed for processing the text portions for extracting the required SVO linguistic features for outputting SVO entity data items according to the invention and/or as the application demands.

For example, once the entities and/or corresponding entity relationships are identified and/or extracted from the text portions of the corpus of text, each of the text segments/portions corresponding to the extracted/identified entities and corresponding entity relationships, respectively, may be input to an SVO linguistic system such as, by way of example only but not limited to, SVO linguistic system 430 and/or 440, combinations thereof, modifications thereto, and/or as herein described in which the SVO linguistic system is configured to identify, for each text portion, the subject entity, verb portion(s) associated with the entity relationship and object entity by applying a domain mapping to determine the meaning of the relationship. This can be based on an ontology/dictionary approach that contains categorised terms for use in identifying the subject, verb portion(s) and/or object of the text portion in relation to the entities and corresponding entity relationship. The SVO linguistic system may scan text portions and/or documents and identify desired words in order to detect dependencies and extract relationships in this way.

The ontology/dictionary approach may include a dictionary of relational terms and their sign (e.g. stimulate vs suppress), plus direction indications (e.g. “lead to” is directional, whereas “represents” is not), and entity terms associated with the domain(s) of interest and/or as the application demands. These can be used to parse the text portions or documents and identify the Subjects and Objects, categorisation information for terms or phrases related to context, and other mappings of terms of interest plus how they apply to the entity relationship. The ontology/dictionary approach may be based on rule-based and/or off-the-shelf NLP and/or linguistic techniques that may be called using an API that implements the SVO linguistic system.

Alternatively or additionally, the SVP linguistic system may be based on one or more linguistic ML model(s) are trained using one or more ML technique(s) and suitable sets of training datasets for identifying and extracting the subject, verb portion(s) and/or object of the text portion in relation to the entities and corresponding entity relationship, and/or meta-data and the like. Alternatively or additionally, a hybrid SVO linguistic system based on one, or both, of these types of systems including an ML model to detect terms within the text portions to be analysed and the like. In relation to ML models, the training datasets can originate from human-annotated data, or from a system with an ML model that learns new terms associated with one or more selected domains of interest and/or associated with one or more selected entity types modal. This system can then be referred to in order to categorise the term and extract the desired information relating entities across a corpus of text, or to output many entities and define the potential entity relationship in terms of, without limitation, for example data representative of sign and direction, supplemented with context data where available.

The SVO process(es) and/or system(s) as described herein may perform the SVO processing and outputting SVO entity data items on a plurality of text portions from a corpus of text. This can be performed in bulk, with a large number of terms and extensive set of textual data, or for a subset of terms such that a particular pair of entities can be investigated for e.g. further study, data set cleansing to remove spurious/non-relations, prioritisation by relationship types.

FIG. 4e is a schematic diagram illustrating an exemplary SVO entity data item 460 and knowledge graph representation for that SVO entity data item 460 for storing the SVO entity data item 460 and/or displaying the SVO entity data item 460 and the like. Data representative of the SVO entity data item 460 may be input to and/or output from an SVO database or repository according to the invention, for example, the SVO entity data items may also be stored as one or more database records. In particular, an SVO entity data item includes data representative of a subject entity 462 is being linked to an object entity 466 via a verb portion 464 associated with the entity relationship, and meta-data associated with the entity relationship. The SVO data item may be stored as a SVO triple including data representative of the subject entity 462, verb portion 464, and object entity 466. FIG. 4e illustrates the text portion of a sentence/statement “Hydroxychloroquine can not reduce cancer risk in pSS patients” as used in previous examples is described for illustrative purposes only. This sentence includes a first entity “Hydroxychloroquine”, a second entity “cancer” and an entity relationship “can not reduce . . . risk in pSS patients.” When analysed by an SVO linguistic system as described with reference to FIGS. 4a to 4d, the first entity may be identified as the subject entity “hydroxychloroquine” (e.g. HCG) 462 and is determined to be linked to the second entity, which is identified as the object entity “cancer” 466, via the identified verb portion 464 of the entity relationship “NOT reduce”. From this, the SVO linguistic system may determine meta-data representing, without limitation, for example a sign/entity sign and direction of the entity relationship linking the entities of the SVO entity data item 462, 464, 466. In this example, the first entity is represented by subject entity node “HCG 468, and the second entity is represented by object entity node “cancer” 472, and a relationship edge 470 linking the subject entity with the object entity is provided and embedded with enhanced relationship information (or meta-data) including the verb portion “NOT reduce” and in which the direction of the relationship is indicated by an arrow on the relationship edge 470 from HCG node 468 to cancer node 466. The sign indication is shown using capital “NOT” in the verb portion, in which “reduce” is negated by “NOT”. The subject entity, verb portion, and object entity may form an SVO triple, with the sign and direction indications forming part of the meta-data associated with the SVO triples. Additionally or alternatively, an SVO entity data item may be formed including data representative of the subject entity, verb portion, object entity, and meta-data associated with the, without limitation, for example, sign, direction, and/or other contextual information associated with the entity relationship corresponding to the subject and/or object entities and the like.

The meta-data associated with the SVO entity data items for each of the received text portions may be based on determining data representative of one or more from the group of: an indication of the direction of the identified relationship between said at least two entities based on identified subject and object entities; biological sign, if any, of the identified relationship between said at least two entities based on identified subject and object entities; affirmation or negation information associated with the identified relationship associated with the between said at least two entities based on identified subject and object entities; context information associated with the identified relationship between the at least two identified entities based on identified subject and object entities; and any other contextual data associated with the relationship between one or more of the at least two identified entities, identified subject entity, identified object entity, verb portion and/or direction.

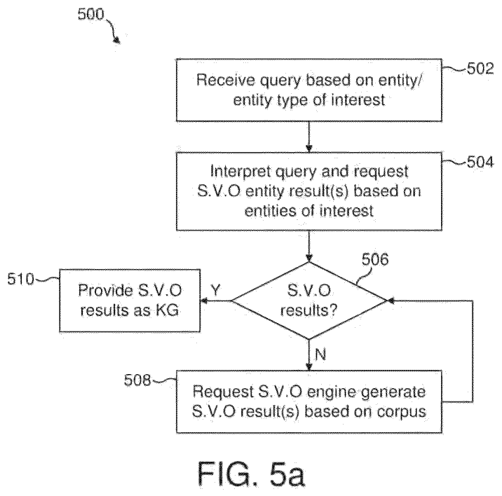

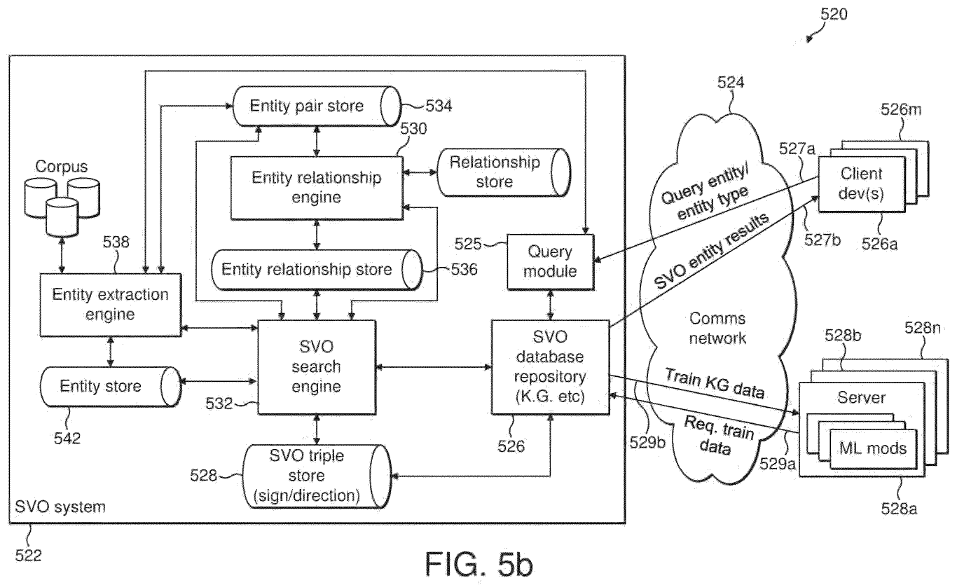

FIG. 5a is a flow diagram illustrating an SVO search process 500 in relation to the SVO search system of FIG. 5b according to the invention. The SVO search process 500 may use the SVO process(es), extraction system(s), and/or SVO linguistic process(es) as described with reference to FIGS. 1a to 4e and/or 5b to 7c, combinations thereof, modifications thereof and/or as described herein, that uses a knowledge graph search index derived from one or more output graph structure(s) associated with SVO entity data items, which may be parsed and/or updated during processing of a search query associated with one or more entities corresponding to one or more domains of interest, and/or one or more entity concepts associated with one or more domains of interest and the like. The SVO search process 500 may include the following steps of: In step 502, the SVO search process receives a search query based on, without limitation, for example data representative of an entity/entity type of interest or associated with one or more domain(s) of interest and the like. In step 504, the SVO search process 500 interprets the query and request SVO entity result(s) based on the entities of interest derived from the interpreted search query. In step 506, if there are positive SVO results denoted by “Y” in the FIG. 5a, then the process 500 proceeds to step 510 in which the SVO results are formed into an SVO results knowledge graph and output by the SVO search system as the formed SVO results knowledge graph. This may be sent to the user associated with the search query and displayed as the SVO results knowledge graph in which entity nodes are linked by entity relationship edges and where each entity relationship edge may be embedded with verb portions and/or meta-data associated with the entity relationship. This is an efficient and concise method for displaying search results compared with conventional tabulated result table formats and the like. The user may instantly understand relationships between entities, the types of relationships between entities and the like.

In step 506, if there are no positive or very small amount of SVO results generated from the currently stored SVO search index knowledge graph, that is if there are no SVO results and/or the SVO search index is out-of-date denoted by “N”, then the process 500 proceeds to step 508 in which a request an SVO engine to generate SVO entity result(s) based on the corpus of text and/or a large scale dataset. The corpus of text and/or large scale dataset may be routinely updated with the latest documents, articles, patent applications, and/or any other content associated with one or more domains of interest. The SVO engine may implement the SVO process(es) according to the invention, in particular, the SVO process(es) as described with reference to FIGS. 1a to 1 e using the process(es) and/or system(s) as described with reference to FIGS. 2a to 4e. These may be used to process portions of text from the corpus of text associated with one or more domains of interest and to output data representative of a set of SVO entity data items. The set of SVO entity data items may be represented as a graph structure with entity nodes being linked by relationship edges, in which meta-data and/or enhanced relationship information such as, without limitation, for example sign of the relationship, verb portions of the entity relationship, and/or direction of the relationship may be embedded in each relationship edge linking one or more entities and the like. The process 500 then proceeds to either steps 504 and/or 506, in which, if there are any new SVO entity results from the output set of SVO data items, these may be included into the SVO search index knowledge graph, and the search query performed again. The process 500 then proceeds to step 506.

As an example, the SVO entity result(s) generated in step 508 based on the text corpus is fed back to determine whether these SVO entity result(s) are new and/or useful for updating the SVO search index graph structure. To make this determination, the SVO search process 500 may query the graph structure for determining whether SVO data items exist in the graph structure associated with the search query. If SVO entity data items exist, then generate a knowledge sub-graph associated with the plurality of entities based on either: SVO entity data items output from the graph structure in relation to the search query, or by filtering the SVO knowledge graph based on the search query. Alternatively, if SVO entity data items in relation to the search query are non-existent or are out-of-date, the steps of receiving portions of text from the corpus of text is performed, which identifies SVO entity data items, and outputting/storing data representative of the sets of SVO entity data items for updating the graph structure.

Alternatively or additionally, the SVO entity data items or the data representative thereof either pre or post storage (storing the SVO entity data items) may be validated. The validation may check for accuracy and resolve conflict and/or aggregation of the plurality of identified SVO entity data item(s) for input to an SVO search index data structure based on one or more from the group of: new SVO entity data items; any contradicting SVO entity data items; multiple identical SVO entity data items that are the same; multiple SVO data items with identical first and second entities with different relationships. The validation may be performed by assessing the number (frequency) of occurrences between two contradicting relationships pertaining to a verb portion within the same the SVO entity data item. In other words, while SVO data items provide the relationships, probability of occurrences could be estimated by how “precedented” a relationship is in the corpus of text (how many number of occurrences you get for the same relationships/aggregates thereof). In turn, the probability may be further accessed downstream or otherwise by a system/process that further assess the probability. In the case of aggregates or aggregation, similar verbs of the SVO data items may be grouped together to give a unified or collective meaning. This could be accomplished using one or more ML models herein described and/or one or more sets of predetermined rules that are associated with one or more domains of interest.

Furthermore, to check for accuracy/validation in regards to entities of interest, SVO entity data item (entity concepts, and/or entity relationships) may be applied for iteratively scanning the corpus of text; based on the number of occurrences, the interested entities could be validated. On the other hand, other search queries such as a distribution-based queries may be used for all the verbs/sign/directionality or contexts or combination of the any two that connect them (and how often each of them does so) to identify the most representative for the interaction (entity) pair. For such entity pair with a relationship, confidence level based on the distribution may be derived for purpose of validation.

In one example, the conflicting relationships may be stored and analysed downstream by a ML model such as one or more ML models herein described. The ML model may assess the similarity (both syntactically and semantically) between the conflicted verbs and group them in a meaningful manner to avoid duplication at pre or post storage stage. The ML model could also assess the context beyond the SVO triple of the SVO entity data item.

For example, to explain the conflicts in the practical case where there are two sentences: 1) gene1 upregulates gene2 in tissue A and 2) gene1 downregulates gene2 in tissue B. From these two sentences, gene1 and gene2 have conflicting verbs, the contextual explanation of the conflict could be that gene1 and gene2 encode for different tissues. The advantage of using contextual information associated with the SVO triple of SVO entity data item would be to further distinguish between relationships amongst SVO entity data items by providing provide additional information to disambiguate these relationships.

In essence, the search query may include data representative of one or more entities, process(es), and/or relationships thereto associated with one or more domain(s) of interest. The relevant set of nodes and/or edges of the search index graph structure may be identified in response to the search query. If SVO entity data items exist in the search index graph structure in relation to the identified nodes and/or edges, then a knowledge sub-graph associated with the plurality of entities based on either: SVO entity data items output from the graph structure in relation to the search query; or filtering the SVO knowledge graph based on the search query may be generated. The sub-graph of the graph structure based on the relevant set of nodes and/or edges may be outputted. Alternatively, in response to determining SVO entity data items in relation to the search query are non-existent or are out-of-date, then performing the steps of receiving portions of text from the corpus of text, identifying SVO entity data items, and outputting/storing data representative of the sets of SVO entity data items for updating the graph structure.

A search algorithm may be used when performing the search query over the search index graph structure, which is built and/or updated using the SVO entity data items output from the SVO process(es) as described with reference to FIGS. 1a to 1e and/or as described herein. The search algorithm may be accustomed to parsing the SVO search index graph data structure based on searching that is dependent on the underlying graph data structures or heuristics. The search algorithms may include but not limited to linear search, greedy (binary) search, digital search, and probabilistic searches such as Grover's algorithm. The various search algorithm(s) may be used in conjunction with or to supplement the use of ML technique(s) for performing a search over the SVO search index graph data structure or any other data structures suitable for storing the SVO entity data items in a search index.

Applicable ML technique(s) may include but are not limited to neural network (NN) structures, tree/graph-based classifiers, linear models and the like and/or any ML technique suitable for modelling/operating on the set of embeddings and/or an embedding vocabulary dataset generated during the training of an ML model(s) or classifier(s). The set of embeddings and/or an embedding vocabulary dataset are generated in relation to on the SVO entity data items, in particular, the SVO triples and/or any associated meta-data may be used as labelled training dataset for one or more ML model(s) through applying the training ML techniques.

FIG. 5b is a schematic diagram illustrating an example search system 520 based on SVO search process 500 describes with reference to FIG. 5a according to the invention, combinations thereof, modifications thereto and/or as herein described. The diagram illustrates an SVO system 522 and its interaction over a communication network 524 with one or more server(s) 528a-528n and/or client device (s) 526a-526n and the like. The SVO system 522 includes a query module 525 is configured for receiving a search query 527a from, in this example, client device 526a, where the search query 527a includes data representative of one or more entities, entity concepts, and/or entity relationships associated with one or more domains of interest and the like. The search query 527a may include data representative of one or more entities associated with the domain(s) of interest. The query module 525 may be coupled to an SVO database or store 526 within which is stored an SVO search index data structure such as, without limitation, for example, an SVO search index knowledge graph and the like. The SVO search index data structure is built and/or updated from SVO entity data items output from the SVO search engine 532 after processing a plurality of text portions from a corpus of text. The SVO search engine 532 is configured to implement one or more SVO process(es) as described with reference to FIGS. 1a to 1e according to the invention using ML models and/or system(s) as described with reference to FIGS. 2a to 5a, combinations thereof, modifications thereof and/or as described herein and the like. The search query may be input to the SVO database 526 for searching the SVO search index data structure based on the search query. The SVO database 526 may output SVO entity results, which may be in the form of a graph structure as described herein. Should a server 528a submit a search query 529a for generating/requesting a labelled training dataset for training one or more ML models, then the SVO search index data structure may be searched and/or parsed in relation to the search query 529a and output SVO results 529b in the form of a labelled training dataset, which may be a labelled graph structure may be sent to the server 526a. The search query may also comprise query entity/entity types. The client device(s) 526a-526m and/or server(s) 528a-528n receive SVO entity result(s) 527b and/or 529b from SVO database/repository 526. This is accomplished via the communication network 524.

In particular, the query module 525 receives the search query 527a for generating SVO entity data item(s) that may comprise a plurality of SVO entity data item(s) from either the graph structure and/or performing the steps of receiving portions of text from the corpus of text via entity extraction engine 538. The entity extraction engine 538 is configured for detecting and extracting a portion of text including at least two entities corresponding to the one or more domain(s) of interest and an entity dependency relationship therebetween. Then, the entity extraction engine 538 outputs entity extraction search results comprising data representative of the extracted portion of text comprising at least two identified entities and the relationship therebetween. More specifically, the steps of receiving portions of text includes: identifying, from the corpus of text, candidate portions of text including one or more entities of interest corresponding to the domain(s) of interest; detecting the most likely candidate portions of text containing at least two entities and an entity dependency relationship therebetween; extracting data representative of the detected entities and relationships therebetween from the detected candidate portions of text; and outputting data representative of entity search results based on the extracted data representative of entities and relationships therebetween. In effect, the entity extraction engine 538 may store the entities extracted as entity store 542 or entity pair store 534, and/or interacting with the query module 525 and/or with SVO search engine 532.

Further in FIG. 5b, the SVO search engine 532 identifies the SVO entity data items from the extracted text portions, and may output and store data representative of the sets of SVO entity data items associated with one or more text portions in SVO database/repository 526. The received search query 529a may also be used to process SVO entity data items output from the SVO search index data structure in relation to the search query into a labelled training dataset that may be stored in the SVO database/repository 526. The labelled training dataset may be used as an input labelled training dataset for training one or more ML model(s) associated with predicting or classifying objective problems and/or processes in the field of: biology, biochemistry, chemistry, medicine, chem(o)informatics, bioinformatics, pharmacology, and any other field relevant to diagnostic, treatment, and/or drug discovery and the like. In turn, the SVO entity data items as a labelled training dataset may be sent in response to the request from the SVO system via the SVO database/repository 526. As such, the one or more servers comprising the one or more ML model(s) may request training data from the database/repository 526 while obtaining training knowledge graph data from the database/repository 526.

FIG. 5b also illustrates the SVO search engine 532 interacting with various other storages such as entity store 542, entity pair store 534, SVO triple store (sign/direction) 528, relationship store, and entity relationship store 536 via the entity relationship engine or process 530. In particular, the relationship store, and entity relationship store 536, and the entity relationship engine may be used by the entity relationship engine or process 530 for identifying, extracting entity relationships from extracted text portions received from SVO search engine 532 and the like. Entity relationship engine or process 530 parses each received portion of text to detect linguistic features. Alternatively or additionally, the entity relationship engine or process 530 be coupled to a linguistic detection engine of SVO search engine, which is coupled to an entity repository 542 and/or entity pair store 534 and an entity relationship repository or store 536, where the linguistic detection engine is configured to use one or more entity repositories 542, 534 and the like in the domain(s) of interest and entity relationship repositories 536 to process said each received portion of text. In effect, the linguistic features in each received portion of text associated with a first entity and a second entity of at least two entities and the entity dependency relationship therebetween are been detected; and the first entity as the subject, the second entity as the object and a segment of the entity dependency relationship as the verb of said each received portion of text are been identified. This is advantageous in that the entities and/or relationships are capable of being detected and/or identified via linguistic detection engine and/or entity relationship search engine 530 while the extracted information may be simultaneously fed into the SVO data based through the SVO search engine 532. This allows the SVO system 520 flexibly adapting to critical changes in real-time.

In operation, a search query 529a for generating SVO entity data item(s) may be received by the query module 525. The SVO system 520 generates SVO entity data item(s) as data representative of an SVO knowledge graph via the portions of text from the corpus of text, identifying SVO entity data items, and outputting/storing data representative of the sets of SVO entity data items. The SVO system 520 sends data representative of the generated SVO knowledge graph in response to the search query 529a for identifying at least one from the group of: new relationships between entity pairs in the domain(s) of interest; new avenues of research associated with entity pairs in the domain(s) of interest.

More specifically, the query module 525 may be configured to receive a plurality of portions of text from the corpus of text with each portion of text comprising data representative of at least two entities and/or relationships thereto, that is extracted using the entity extraction engine 538 and/or entity relationship engine 530. In addition, an SVO search engine 532 is configured to receive the portions of text and identify, for each received portion of text, one or more SVO entity data items comprising data representative of at least two entities, a relationship associated with the at least two entities, a subject entity corresponding to an entity of the at least two entities, an object entity corresponding to an entity of the at least two entities, a verb portion associated with the relationship, and a sign or direction of the relationship associated with the at least two entities. The SVO search engine 532 may interact with the entity extraction engine 538 and/or entity relationship engine 530. Finally, an output module that may be coupled to the SVO database/repository 526 may be configured to output a set of identified SVO entity data items for use in building a graph search index for the SVO database/repository 526. The graph search index including a graph of entity nodes with relationship edges between each entity and an indication of the verb portion and directionality associated with each relationship between entities.

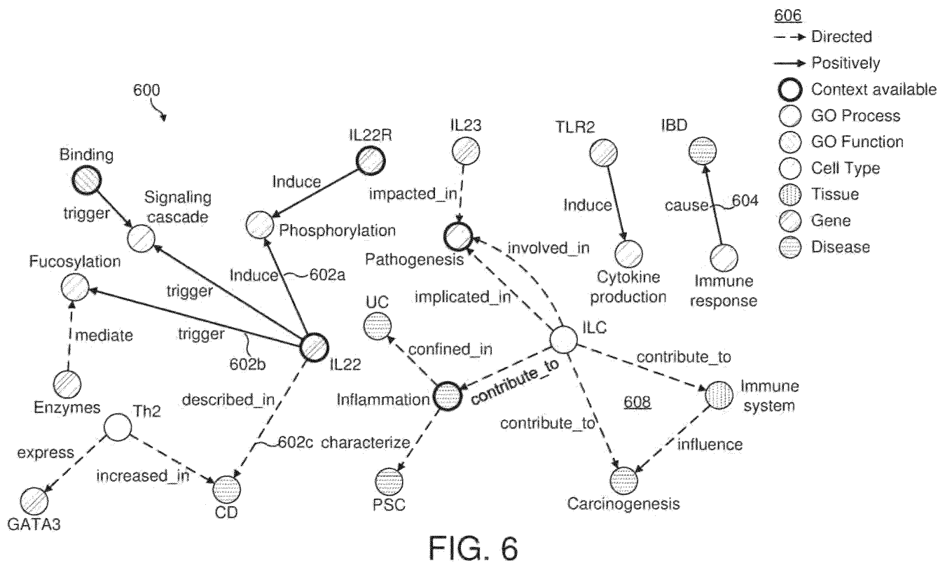

FIG. 6 is a schematic diagram illustrating an exemplary SVO knowledge graph 600 generated from a corpus of text associated with several domains of interest. Although the domains of interest generally fall within the biological and/or chemical domains (or bioinformatics and/or chem(o)informatics domains), in this example, there are at least six domains of interest that include, without limitation, for example, genes, diseases, tissues, cell-types, gene ontology (GO) biological process (GO Process), and GO biological function (GO Function). In this example, the SVO process(es) as described with reference to FIGS. 1a to 1e using one or more process(es), system(s) and/or apparatus as described with reference to FIGS. 2a to 5b and the like according to the invention may process text portions from a corpus of text associated with the above at least six domains of interest to generate and output data representative of a set of SVO entity data items associated with these at least six domains of interest. The entities associated with the SVO data items include entities associated with the six domains of interest and include entities with entity types including, without limitation, for example one or more from the group of: disease, gene, tissue, cell-type, GO process, and GO function and the like. Furthermore, the data representative of the set of SVO entity data items may be output in the form of SVO knowledge graph 600, which may be, without limitation, for example displayed to a user, used by a server for training one or more ML model(s), and/or used for building a search index graph data structure for the search system 520 of FIG. 5b and the like, and/or used as the application demands. Once the entities and entity relationships thereto from text portions of a corpus of text associated with the above six domains of interest are identified, extracted and linguistically processed to form a set of SVO entity data items as described herein with reference to FIGS. 1a to 5b, the set of SVO entity data items may be processed to form the SVO knowledge graph 600 (or domain map 600) as illustrated in FIG. 6.

The SVO knowledge graph 600 includes a plurality of nodes representing entities associated with the domains of interest such as, in this example, without limitation, entity types from the group of: drugs, diseases, gene, tissue, cell-type GO process, GO function etc.

The edges between entity nodes represent entity relationships between, for example, drug and the disease entity nodes or relationships between an entity node of a particular entity type or domain and another entity node of another particular entity type or domain. The legend 606 of FIG. 6 highlights the colouring/hashing/shading of various entity nodes (coloured/shaded/hashed circles), where each colour/shade/hash of a node suggests its domain and/or entity type that may include, by way of example only but is not limited to, “GO process” depicted as circles with left spaced apart diagonal hashing; “GO function” depicted as circles with right diagonal hashing; “cell type” depicted as circles with single left diagonal line; “tissue” depicted as circles with vertical hashing; “gene” depicted as circles with left close-spaced diagonal hashing; “disease” depicted as circles with horizontal hashing; and context available depicted as bold outlined circles. In this example, the “immune response” node is typed as a GO process entity while “IBD” node is typed as a disease entity. In effect, the colouring/shading/hashing of the entity nodes suggests its entity type or the relative type in relation to the other entity nodes. As well, the sign or biological sign and the direction of the relationship between nodes are respectively depicted by the shading/hashing/colouring/line-type of the relationship edge arrows between entity nodes and the direction of these arrows. That is, the direction of the arrows indicates the direction of the relationship, the verb portion indicated as text embedded on an arrow relates to the relationship, and line-type may indicate biological sign/entity sign/sign of the relationship including, without limitation, for example a positive sign (“positively”), negative sign (“negatively”), directed sign (“directed”) and the like. The contextual information of the relationship edges between entity nodes are embedded with verb portions and represented as outlined text e.g. “contriubute_to”, “influence”, “trigger, “described_in”. For example, a solid arrow 602a from “IL22” node to “phosphorylation” node depicts a “positively” sign underlying verb portion “induce” of the relationship with the direction of the relationship stemming from “IL22” node, similarly a solid arrow 602b from “IL22” node to “Fucosylation” node depicts a “positively” sign underlying verb portion “trigger” of the relationship with the direction of the relationship stemming from “IL22” node. In another example, a dashed arrow 602c from “IL22” node to “CD” node depicts a “directed” sign underlying verb portion “described_in” of the relationship with the direction stemming from “IL22” node.

By iterating over many nodes/edges of the graph relationship may be aggregated or amalgamated 608 to estimate the sign and direction or any other derivable meta-data. For example, starting from “ILC” node that contributes to “immune system” node in turn influences “carcinogenesis” node, which may also be arrived directly from “ILC” node. As such, the graph may be traversed iteratively as to estimate the sign and direction by aggregating or amalgamating, the biological sign indications associated with the two or more identified SVO entity data item(s) to determine an overall biological sign and direction. Alternatively or additionally, the edges between other entities nodes (not shown in the figure) may be also represent entity relationships amongst any such two entities selected from, without limitation, for example a group of: disease, drug, protein, gene, and the like. For example, the relationships amongst any such two entities may be tissue-cell type, organ-cell line, disease-species, and the like.

In one example, the graph or domain map may be derived from using an ontology/dictionary described herein, which is provided to contain categorised terminology that may be labelled using SVO entity data items. In particular, the SVO entity data items may be represented as SVO triples and associated meta-data such as sign and direction may be used in conjunction with an NLP system. The NLP system may be used to categorise and identify possible terminology for use in generating the graph/domain map that maps one to one relationships, one to many relationships, and many to one relationships. The domain map, in turn, permits rapid reviewing of documents and identify desired terminology for the purpose of detecting dependences entity relationships and extract these entity relationships efficiently. In effect, the terminology and the desired information relating entities across a corpus of text may be extracted in bulk; or entities may be used to define the potential relationship in terms of sign and direction may be sorted and searched.

In one example, a graph/domain mapping engine may use one or more ontologies/dictionaries, where the ontologies can contain a dictionary of relational terms and their sign (e.g. stimulate vs suppress), plus direction (e.g. “lead to” is directional, whereas “represents” is not), and entity terms of interest. Using the sign and direction or other meta-data or mapping terms, such a domain mapping engine may in turn provide a data structure where the NLP system smoothly appropriates a desired word or relationship from text portions of documents of a corpus of text including unstructured data and the like.

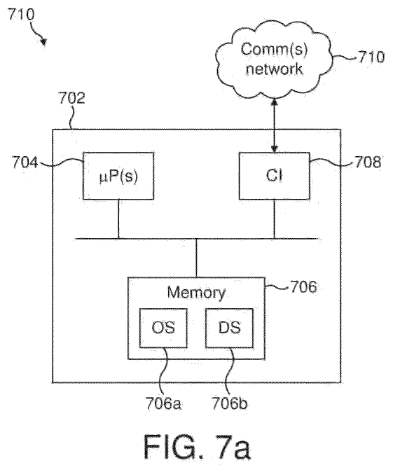

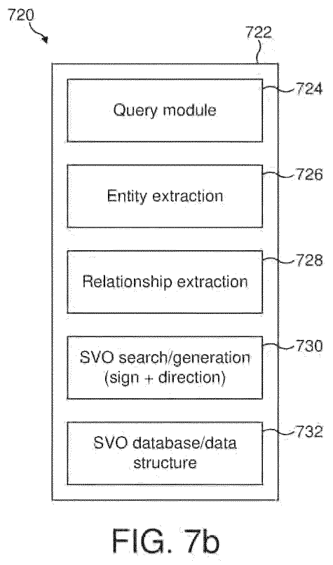

FIG. 7a is a schematic diagram is a schematic diagram illustrating a computing system 700 including a computing device, server and/or apparatus 702 coupled to a communications network 710 that may be used to implement one or more aspects of the SVO process(es) and/or other aspects according to the invention and/or implement one or more method(s), process(es), ML model(s), and/or system(s) and apparatus as described with reference to FIGS. 1a-7b according to the invention. Computing device 702 includes one or more processor unit(s) (μPs) 704, memory unit 706 and communication interface (CI) 708 in which the one or more processor unit(s) 704 are connected to the memory unit 706 and the communication interface 710. The communications interface 710 may connect the computing device 702 with one or more databases or other processing system(s) or computing device(s)/server(s) via communications network 710. The memory unit 706 may store one or more program instructions, code or components such as, by way of example only but not limited to, an operating system (OS) 706a for operating computing device 702 and a data store (DS) 706b for storing additional data and/or further program instructions, code and/or components associated with implementing the functionality and/or one or more function(s) or functionality associated with one or more of the method(s) and/or process(es) of the apparatus, module(s), ML model(s), systems(s), mechanisms and/or system(s)/platforms/architectures as described herein and/or as described with reference to at least one of figure(s) 1a to 7b.

Further aspects of the invention may include one or more apparatus and/or devices that include a communications interface, a memory unit, and a processor unit, the processor unit connected to the communications interface and the memory unit, wherein the processor unit, storage unit, communications interface are configured to perform the system(s), apparatus, method(s) and/or process(es) or combinations thereof as described herein with reference to any one of FIGS. 1a to 7b.

FIG. 7b is a schematic diagram illustrating a 720 system according to the invention. The system comprises a general SVO system 722 comprising a query module 724, an entity extraction 726, relationship extraction 728, SVO search/generation 730, and SVO database/data structure 732. The query module may be part of the input module or is independently configured to receive a plurality of portions of text from the corpus of text, each portion of text comprising data representative of at least two entities and/or relationships thereto. The entity extraction 726, relationship extraction 728, and SVO search/generation 730 may be part of an SVO engine or are independently or combinatorially configured to identify, for each received portion of text, one or more SVO entity data items comprising data representative of at least two entities, a relationship associated with the at least two entities, a subject entity corresponding to an entity of the at least two entities, an object entity corresponding to an entity of the at least two entities, a verb portion associated with the relationship, and a direction of the relationship associated with the at least two entities. SVO database/data structure 732 may be part of the output module or is independently configured to output a set of identified SVO entity data items for use in building a graph search index, the graph search index comprising a graph of entity nodes with relationship edges between each entity and an indication of the verb portion and directionality associated with each relationship between entities. In particular, the query module may be configured for receiving a search query comprising data representative of one or more entities and/or relationships associated with one or more domains of interest, where an SVO search module configured for processing the search query based on an SVO search index data structure. The SVO system may 720 build or update the SVO search index data structure based on an output set of SVO entity data items. The system 720 may include the functionality of the method(s), process(es), and/or system(s) associated with the invention as described herein, or as described with reference to FIGS. 1a-7a, combinations thereof, modifications thereto and/or as the application demands and the like.

Further aspects of the invention may include one or more apparatus and/or devices that include a communications interface, a memory unit, and a processor unit, the processor unit connected to the communications interface and the memory unit, wherein the processor unit, storage unit, communications interface are configured to perform the system(s), apparatus, method(s) and/or process(es); modifications thereof; combinations thereof; as described herein; and/or as described with reference to FIGS. 1a to 7b.

In the embodiment(s) described above the method(s), apparatus, system(s) and/or computing system/device(s) may be implemented by a server, the server may comprise a single server or network of servers. In some examples the functionality of the server may be provided by a network of servers distributed across a geographical area, such as a worldwide distributed network of servers, and a user may be connected to an appropriate one of the network of servers based upon a user location.

The above description discusses embodiments of the invention with reference to a single user for clarity. It will be understood that in practice the system may be shared by a plurality of users, and possibly by a very large number of users simultaneously.

The embodiments described above are fully automatic or semi-automatic. In some examples a user or operator of the system may manually instruct some steps of the method to be carried out.

In the described embodiments of the invention the system may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium. Computer-readable media may include, for example, computer-readable storage media. Computer-readable storage media may include volatile or non-volatile, removable or non-removable media implemented in any method or technology for storage of information such as computer readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Application Program-specific Integrated Circuits (ASICs), Application Program-specific Standard Products (ASSPs), System-on-a-chip systems (SOCs). Complex Programmable Logic Devices (CPLDs), etc.

Although illustrated as a single apparatus or system, it is to be understood that the computing device or system may be a distributed system or part of a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface). Furthermore, the systems, apparatus, and/or method(s) as described herein may be distributed or located remotely and accessed via a network or other communication link (e.g. using a communication interface).

The term ‘computer is used herein to refer to any device with processing capability such that it can execute instructions. Those skilled in the art will realise that such processing capabilities are incorporated into many different devices and therefore the term ‘computer’ includes PCs, servers, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages. Variants should be considered to be included into the scope of the invention.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “module”, “component” and/or “system” are intended to encompass computer-readable data storage that is configured with computer-executable instructions that cause certain functionality to be performed when executed by a processor. The computer-executable instructions may include a routine, a function, or the like. It is also to be understood that a module, component and/or system may be localized on a single device or distributed across several devices.

Further, as used herein, the term “exemplary” is intended to mean “serving as an illustration or example of something”.

Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art. What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible.

Accordingly, the described aspects are intended to embrace all such alterations, modifications, and variations that fall within the scope of the appended claims. Although various embodiments have been described above with a certain degree of particularity, or with reference to one or more individual embodiments, those skilled in the art could make numerous alterations to the disclosed embodiments without departing from the spirit or scope of this invention.

1. A computer-implemented method of automatically extracting entities associated with one or more domain(s) of interest from a corpus of text, the method comprising:
receiving a plurality of portions of text from the corpus of text, each portion of text comprising data representative of at least two entities and/or relationships thereto;
identifying, for each received portion of text, one or more subject-verb-object “SVO” entity data item(s) comprising data representative of at least two entities, a relationship associated with the at least two entities, a subject entity corresponding to an entity of said at least two entities, an object entity corresponding to an entity of the at least two entities, a verb portion associated with the relationship, and a direction of the relationship associated with the at least two entities;
outputting a graph structure based on the set of identified SVO entity data items, the graph structure comprising a graph of entity nodes and relationship edges linking the entity nodes with each relationship edge including an indication of directionality of said relationship.

2. The computer-implemented method as claimed in claim 1, further comprising identifying meta-data from each of the received text portions for inclusion to each SVO entity data item, the meta-data comprising data representative of one or more from the group of:
directionality associated with each relationship;
biological sign or entity sign, where applicable, associated with each relationship;
affirmation or negation information associated with each relationship;
context information associated with each relationship;
any other contextual data associated with said each relationship; and
any other contextual data associated with the directionality and/or biological sign associated with each relationship; and
outputting the graph structure based on the set of identified SVO data items, wherein the relationship edges linking the entity nodes include indications of the one or more identified meta-data from the corresponding SVO entity data item(s) associated with the entity nodes.

3. The computer-implemented method as claimed in claim 1 or 2, wherein each of the at least two entities comprise data representative of a noun or a noun phrase associated with the one or more domains of interest, and wherein the subject entity corresponds to a first noun or a first noun phrase and the object entity corresponds to a second noun or a second noun phrase.

4. The computer-implemented method as claimed in any preceding claim, wherein each entity of the at least two entities is a named entity from an entity dictionary associated with at least one of the domain(s) of interest, and identifying one or more SVO entity data items further comprises identifying the first and second entities as named entities from the portion of text based on one or more entity dictionaries associated with said one or more domains of interest, wherein identifying the first and second entities further comprises performing an entity search of the received portions of text based on the one or more entity dictionaries associated with the one or more domain(s) of interest for identifying data representative of at least two entities associated with the one or more domains of interest and an entity dependency relationship therebetween.

5. The computer-implemented method as claimed in any preceding claim, wherein identifying an SVO entity data item for each received portion of text further comprising performing relationship extraction on said each received text portions to identify at least two entities and an entity dependency relationship therebetween.

6. The computer-implemented method as claimed in claim 9, wherein receiving the plurality of portions of text from the corpus of text, further comprising performing relationship extraction on the received portions of text for at least predicting or identifying at least two entities and an entity dependency relationship thereto.

7. The computer-implemented method as claimed in any preceding claim, wherein receiving the plurality of portions of text from the corpus of text, further comprising:
receiving a plurality of portions of text from the corpus of text; and
detecting, from the received plurality of portions of text, one or more portions of text likely to include at least one entity for use in identifying SVO entity data.

8. The computer-implemented method as claimed in any preceding claim, wherein identifying an SVO entity data item for each of the received portions of text further comprising performing SVO identification on said each received text portions based on identifying:
a subject entity corresponding to an entity of the at least two identified entities;
an object entity corresponding to an entity of the at least two identified entities; and
a verb portion associated with the identified relationship.

9. The computer-implemented method as claimed in any preceding claim, wherein performing SVO identification further comprises:
detecting linguistic features of the from each of the received portions of text that connect the at least two identified entities;
extracting data representative of the subject entity, object entity, verb portions, and direction based on the at least two identified entities; and
adding the extracted direction indication to the relationship associated with the at least two entities.

10. The computer-implemented method as claimed in any preceding claim, wherein performing SVO identification for each received portion of text further comprising:
detecting linguistic features from one or more segments of text of the received portion of text that connect the at least two identified entities; and
extracting data representative of the subject entity, object entity, verb portions, and direction based on the detected linguistic features from said segments and the at least two identified entities.

11. The computer-implemented method as claimed in any preceding claim, wherein identifying SVO data items(s) further comprising:
performing SVO entity identification on each of the received text portions based on identifying a subject entity, an object entity, and a verb entity associated with a relationship between the identified subject entity and the identified object entity;
performing relationship extraction on each of the received text portions to identify at least two entities and an entity dependency relationship therebetween; and
associating the subject entity with one of the at least two identified entities, the object entity with one of the at least two identified entities, and the verb entity identifying an entity of the at least two identified entities to the subject-entity.

12. The computer-implemented method as claimed in any preceding claim, wherein identifying, from each of the received portions of text, SVO entity data representative of at least two entities and a relationship associated with the at least two entities further comprising
inputting each received portion of text into a relationship extraction model configured for predicting or identifying at least two entities and a relationship therebetween for said each received portion of text.

13. The computer-implemented method as claimed in any preceding claim, wherein identifying, from each of the received portions of text, SVO entity data representative of a subject entity corresponding to an entity of the at least two entities, an object entity corresponding to an entity of the at least two entities, a verb portion associated with the relationship, further comprising:
inputting at least two entities and a relationship therebetween in relation to each received portion of text into a SVO extraction model configured for predicting or identifying a subject entity corresponding to an entity of the at least two entities, an object entity corresponding to an entity of the at least two entities, a verb portion associated with the relationship therebetween for said each received portion of text.

14. The computer-implemented method as claimed in any preceding claim, wherein identifying, from each of the received portions of text, SVO entity data item(s) further comprising:
inputting each received portion of text into a SVO identification model configured for predicting or identifying a subject entity corresponding to an entity of the at least two entities, an object entity corresponding to an entity of the at least two entities, a verb portion associated with the relationship therebetween for said each received portion of text.

15. The computer-implemented method as claimed in any preceding claim, wherein the domain of interest includes biological and/or chemical domains of interest and the entities have entity types in the domain of biological and/or chemical domains.

16. The computer-implemented method as claimed in any preceding claim, wherein:
identifying, for each of the received portions of text, an SVO entity data item further comprising:
identifying one or more SVO triples based on the at least two entities and an entity dependency relationship therebetween, wherein the subject of one of the SVO triples is associated with a first entity of the at least two entities, the object of said one of the SVO triples is associated with a second entity of the at least two entities, and the verb of said one of the SVO triples is associated with the entity dependency relationship between the first and second entities; and
determining, for each identified SVO triple, meta-data representative of at least the direction of the entity dependency relationship between the first and second entities corresponding to said each SVO triple; and
outputting an SVO entity data item comprising data representative of the identified SVO triple and at least the direction of the entity dependency relationship between the first and second entities of said identified SVO triple.

17. The computer-implemented method as claimed in any preceding claim, wherein identifying an SVO entity data item for each of the received portions of text further comprising:
inputting said each received portion of text into an entity extraction engine or process configured for detecting and extracting a portion of text including at least two entities corresponding to the one or more domain(s) of interest and an entity dependency relationship therebetween; and
outputting entity extraction search results comprising data representative of the extracted portion of text comprising at least two identified entities and the relationship therebetween.

18. The computer-implemented method as claimed in claim 17, wherein the entity extraction engine or process is configured to perform the steps of:
identifying, from the corpus of text, candidate portions of text including one or more entities of interest corresponding to the domain(s) of interest;
detecting the most likely candidate portions of text containing at least two entities and an entity relationship therebetween;
extracting data representative of the detected entities and relationships therebetween from the detected candidate portions of text; and
outputting data representative of entity search results based on the extracted data representative of entities and relationships therebetween.

19. The computer-implemented method as claimed in claim 18, wherein detecting the most likely candidate portions of text further comprises parsing each identified candidate portion of text to determine whether an entity relationship exists in relation to the one or more entities.

20. The computer-implemented method as claimed in any of claim 17 or 18, wherein the entity extraction engine or process comprises an entity extraction machine learning model configured to identify, predict, detect and/or extract portions of text comprising at least two entities associated with the one or more domains of interest and a relationship therebetween from a corpus of text or documents.

21. The computer-implemented method as claimed in claim 20, further comprising:
inputting portions of text from the corpus of text associated with the one or more domain(s) of interest to one or more machine learning, ML, extraction model(s) configured for identifying and/or predicting whether the portions of text include at least two entities in one or more domain(s) of interest and an entity dependency relationship therebetween.

22. The computer-implemented method as claimed in any of claim 20, further comprising:
inputting portions of text determined to include one or more entity(ies) associated with one or more domain(s) of interest to one or more machine learning, ML, extraction model(s) configured for identifying and predicting whether a portion of text with one or more entity(ies) of interest forms at least two entities and an entity dependency relationship therebetween.

23. The computer-implemented method as claimed in any of claims 17 to 22, wherein the entity extraction engine or process further comprises a rule-based engine or process configured to:
identify, from the received portions of text of the corpus of text, text portions including one or more entity(ies) associated with the one or more domains of interest based an entity search of the received portions of text using on one or more entity dictionaries associated with the one or more domains of interest; and
extracting, from each identified text portion, data representative of at least two entities associated with the one or more domains of interest and an entity relationship therebetween.

24. The computer-implemented method as claimed in any of the preceding claims, wherein the step of identifying, for each of the received portions of text, one or more SVO entity data item(s) further comprising:
parsing said each received portion of text for detecting linguistic features associated with the at least two entities associated with the domain(s) of interest and corresponding entity dependency relationship therebetween;
identifying, from said each received portion of text, a first entity of the at least two entities associated with the subject of the received portion of text, a second entity of the at least two entities associated with the object of the received portion of text, and a verb segment of the entity dependency relationship associated with the verb of the identified relationship in the received portion of text; and
outputting a set of SVO entity data items representative of an subject-verb-object triple based on data representative of the first entity, segment of the entity relationship, and the second entity.

25. The computer-implemented method as claimed in claim 33 wherein parsing said each received portion of text for detecting linguistic features further comprising a linguistic detection engine coupled to an entity repository and an entity relationship repository, wherein the linguistic detection engine is configured to use one or more entity repositories in the domain(s) of interest and entity relationship repositories to process said each received portion of text by:
detecting linguistic features in said each received portion of text associated with a first entity and a second entity of at least two entities and the entity dependency relationship therebetween; and
identify the first entity as the subject, the second entity as the object and a segment of the entity dependency relationship as the verb of said each received portion of text.

26. The computer-implemented method as claimed in any preceding claim, further comprising:
determining, for each SVO entity data, at least the biological sign and direction of the entity dependency relationship based on a domain mapping engine coupled to an ontological dictionary of relational terms associated with entities and entity relationships, the domain mapping engine configured for:
determining a segment of the entity relationship representing a biological sign of the entity dependency relationship for the at least two entities of said each SVO entity data item;
determining a direction indication of the entity dependency relationship representing the direction of the entity dependency relationship between the first and second entities of the at least two entities of said each SVO entity data item; and
updating said each SVO entity data item with data representative of the segment representing the biological sign of the entity dependency relationship and data representative of the direction indication of the entity dependency relationship.

27. The computer-implemented method as claimed in claim 26 further comprising:
determining one or more further contextual elements of the entity relationship representing the context of the entity relationship between the first and second entities of the at least two entities of said each SVO entity data item; and
updating said each SVO entity data item representative of the contextual segments.

28. The computer-implemented method as claimed in any preceding claim, further comprising determining, for each identified SVO entity data item, at least the biological sign, and direction of the entity relationship based on:
inputting data representative of a received portion of text associated with the SVO entity data item, the corresponding at least two entities, and/or the corresponding entity relationship, to a domain mapping machine learning model configured to identify or predict a biological sign of the entity dependency relationship for the at least two entities, and to identify or predict a direction indication of the entity relationship representing the direction of the entity relationship between the first and second entities of the at least two entities; and
updating said each SVO entity data item with data representative of the predicted biological sign and direction of the entity relationship.

29. The computer-implemented method as claimed in any preceding claim, further comprising storing data representative of each of the output identified SVO entity data item(s) and corresponding biological sign and direction of the entity relationship based on:
performing validation, conflict resolution and/or aggregation of the plurality of identified SVO entity data item(s) for input to an SVO search index data structure based on one or more from the group of: new SVO entity data items; any contradicting SVO entity data items; multiple identical SVO entity data items that are the same; multiple SVO data items with identical first and second entities with different relationships; and
storing the validated SVO entity data items in the SVO search index data structure for use in outputting SVO search results based on received SVO search queries querying the SVO search index data structure, wherein the SVO search queries comprise data representative of one or more entities, process(es) and/or relationships thereto in the domain(s) of interest.

30. The computer-implemented method as claimed in any preceding claim, further comprising aggregating two or more of the identified SVO entity data items(s) with the same entity pair and similar entity relationship by:
aggregating the biological sign indications associated with the two or more identified SVO entity data item(s) to determine an overall biological sign;
aggregating the direction indications associated with the two or more identified SVO entity data item(s) to determine an overall direction indication;
generating an aggregated SVO entity data item comprising data representative of the entity pair, the entity dependency relationship, and the overall biological sign and overall direction indication; and
storing data representative of the aggregated SVO data item in the SVO search index data structure.

31. The computer-implemented method as claimed in any preceding claim, wherein the SVO search index data structure comprises a graph structure based on the output and/or stored set of SVO entity data item(s).

32. The computer-implemented method as claimed in any preceding claim, wherein set of SVO entity data items comprise a plurality of SVO entity data items, each SVO entity data item associated with data representative of at least an indication of the biological sign and direction of the entity relationship between at least two entities, and the set of SVO entity data items are stored in a graph structure comprising a plurality of nodes linked together by edges, wherein each node of the graph structure represents an entity, and an edge linking a pair of nodes represents a relationship between a pair of entities represented by the pair of nodes, the edge further comprising data representative of an indication of the direction associated with the relationship between the pair of entities.

33. The computer-implemented method as claimed in claim 32, the method further comprising:
receiving a search query comprising data representative of one or more entities, process(es), and/or relationships thereto associated with one or more domain(s) of interest;
querying the graph structure for finding a relevant set of nodes and/or edges associated with the search query, and outputting a sub-graph of the graph structure based on the relevant set of nodes and/or edges associated with the search query.

34. The computer-implemented method as claimed in claim 33, the method further comprising:
querying the graph structure for determining whether SVO data items exist in the graph structure associated with the search query;
in response to determining SVO entity data items exist, generating a knowledge sub-graph associated with the plurality of entities based on one or more of: SVO entity data items output from the graph structure in relation to the search query; filtering the SVO knowledge graph based on the search query;
in response to determining SVO entity data items in relation to the search query are non-existent or are out-of-date, performing the steps of receiving portions of text from the corpus of text, identifying SVO entity data items, and outputting/storing data representative of the sets of SVO entity data items for updating the graph structure.

35. The computer-implemented method as claimed in any of claims 33 to 34, wherein a search query comprises a request for a labelled training dataset associated with entity pairs and relationships thereto associated with domain(s) of interest, wherein the method further comprising:
processing the SVO entity data items output from the SVO search index data structure in relation to the search query into a labelled training dataset, wherein the labelled training dataset is for use as an input labelled training dataset for training one or more ML model(s) associated with predicting or classifying objective problems and/or processes in the field of: biology, biochemistry, chemistry, medicine, chem(o)informatics, bioinformatics, pharmacology, and any other field relevant to diagnostic, treatment, and/or drug discovery and the like; and
sending the processed SVO entity data items as a labelled training dataset in response to the request.

36. The computer-implemented method as claimed in any preceding claim, wherein a biological and/or chemical entity comprises entity data associated with an entity type from at least the group of: gene; disease; compound/drug; protein; cell type; tissue; chemical; organ;
biological parts; mechanisms or systems; or any other entity type associated with bioinformatics, chem(o)informatics, biology, biochemistry, chemistry, medicine, pharmacology, and/or any other field relevant to diagnostic, treatment, and/or drug discovery and the like.

37. A computer-readable medium comprising code or computer instructions stored thereon, which when executed by a processor unit, causes the processor unit to perform the computer-implemented method according to any one of claims 1 to 36.

38. An apparatus comprising a processor unit, a memory unit and a communication interface, the processor unit connected to the memory unit and communication interface, wherein the apparatus is adapted to implement the computer-implemented method according to any one of claims 1 to 37.

39. An SVO apparatus of automatically extracting entities associated with one or more domain(s) of interest from a corpus of text, the system comprising:
an input module configured to receive a plurality of portions of text from the corpus of text, each portion of text comprising data representative of at least two entities and/or relationships thereto;
an SVO engine configured to identify, for each received portion of text, one or more subject-verb-object “SVO” entity data items comprising data representative of at least two entities, a relationship associated with the at least two entities, a subject entity corresponding to an entity of the at least two entities, an object entity corresponding to an entity of the at least two entities, a verb portion associated with the relationship, and a direction of the relationship associated with the at least two entities; and
an output module configured to output a set of identified SVO entity data items.

40. A search system, the system comprising:
a search query module configured for receiving a search query comprising data representative of one or more entities and/or relationships associated with one or more domains of interest;
an SVO search module configured for processing the search query based on an SVO search index data structure; and
an SVO apparatus according to claim 39 configured for building or updating the SVO search index data structure based on an output set of SVO entity data items.

41. The computer-implemented invention, search engine apparatus, apparatus as claimed in any preceding claim, wherein the corpus of text comprises a large scale document repository including a plurality of documents associated with a plurality of domain(s) of interest, biological entity and/or chemical entity concepts; and
the corpus of text further comprising data representative of one or more from the group of: unstructured text, semi-structured text, documents, sections of documents, sentences and/or paragraphs of documents, tables, and/or any portions of text and/or data representative of one or more entities and/or relationships thereto capable of being detected and/or identified using relationship extraction techniques and the like.

42. A computer-implemented method, apparatus or system as claimed in any preceding claim, wherein an entity comprises entity data associated with an entity type in relation to a domain of interest from at least the group of: bioinformatics; chem(o)informatics; data informatics; social media; entertainment; geographical; any other entity type in which a portion of text comprises data representative of a relationship for one or more entity(ies); and
wherein the domain of interest comprises one or more domains or fields associated with an entity type from at least the group of: genes; diseases, disease process(es) or pathway(s); biological part(s), biological process(es) or pathway(s); compound/drug; protein(s); cell-line(s); chemical; tissue; organ; or any other domain of interest or entity type associated with bioinformatics, pharmacology and/or chem(o)informatics and the like.
